(19) United States
(12) Patent Application Publication
(10) Pub. No.: US 2009/0299436 A1
(43) Pub. Date: Dec. 3, 2009

(54) MITIGATION OF PRESSURE ULCERS USING ELECTRICAL STIMULATION

(76) Inventors: Vivian K. MUSHAHWAR, Edmonton (CA); Leandro Rafael SOLIS-AGUILAR, Edmonton (CA)

Correspondence Address: OGILVY RENAULT LLP, 1, Place Ville Marie, SUITE 2500, MONTREAL, QC, H3B 1R1, CA

(21) Appl. No.: 12/362,725

(22) Filed: Jan. 30, 2009

Related U.S. Application Data
(60) Provisional application No. 61/025,472, filed on Feb. 1, 2008.

(51) Int. Cl. A61N 1/36

(52) U.S. Cl. 607/48

(57) ABSTRACT

There is provided a method for mitigating or preventing formation of pressure ulcers in a patient by transmitting an electrical stimulus to a skin portion of a patient sufficient to effect contraction of a muscle, wherein the method includes a first mode of operation and a second mode of operation. The method includes over a period of at least about one hour, continuously switching between the first mode of operation and the second mode of operation. For each instance of the first mode of operation, the first mode of operation lasts a respective predetermined stimulus time duration, and a respective operative electrical stimulus, sufficient to effect contraction of a muscle, is transmitted to the skin portion, thereby effecting contraction of the muscle, during the entire, or substantially the entire, respective predetermined stimulus time duration. For each instance of the second mode of operation, the second mode of operation lasts a respective predetermined relaxation time duration, and the muscle is relaxed during the entire, or substantially the entire, respective predetermined relaxation time duration.

This application claims the benefit of U.S. Provisional Patent Application Ser. No. 61/025,472 filed on Feb. 1, 2008 under 35 U.S.C. 119(e).

### FIELD OF THE INVENTION

This invention relates to the mitigation treatment of pressure ulcers and, in particular, the mitigation of treatment of pressure ulcers through electrical stimulation.

### BACKGROUND OF THE INVENTION

Pressure ulcers are typically associated with individuals of compromised mobility, namely the infirm, the elderly, and people with spinal cord injury (see references 10, 12, 31, 49, 60, 61). A pressure ulcer is any lesion caused by unrelieved pressure resulting in damage of underlying tissue (see reference 1), involving any one of, or any combination of, skin, fat, fascia, muscle, or bone. Pressure ulcers develop following a prolonged period of compression of the tissue between a bony prominence and a surface (see references 13, 24, 48, 53, 60) which causes the occlusion of capillaries and leads to ischemia. Ischemia, therefore, has historically been considered a major factor leading to pressure ulcer formation (see references 27-29). Paradoxically, the restoration of blood flow, vital to preserving tissue viability, has also been identified to cause extended damage of the tissue (see references 20, 23, 41, 55). In instances where the ischemic state has been maintained for extended periods, the influx of oxygen-rich blood causes the activation of free radicals, further damaging the cells in the tissue (see references 20, 23, 41, 55). In addition to the injury caused by biochemical changes occurring during tissue ischemia and ensuing reperfusion, high stress levels at the bone-muscle interface and the duration of their application, have also been reported as direct causes of tissue injury (see references 7, 8, 11, 35-37). Furthermore, injury to the muscle results in the formation of scar tissue; thus, creating more foci for increased stress, and leading to injury of adjacent previously healthy tissue (see references 18, 36). It is the combined effects of these processes that cause the edema, inflammation and necrosis that ultimately lead to formation of a pressure ulcer (see references 14, 19, 20, 47, 56, 57).

Pressure ulcers can be initiated at the dermis, usually in the presence of excessive friction and/or compromised dermal integrity and progress towards the deeper layers of tissue. Muscle is considered to be more susceptible to tissue degradation from mechanical loading and oxygen deprivation (see references 7, 31) than dermis, consequently injury can also be induced in the deep tissue and progress outwards (see reference 11), evolving into a severe full-thickness pressure ulcer. This type of pressure-related injury to the deep tissue under intact skin has been defined by the National Pressure Ulcer Advisory Panel as deep tissue injury (DTI) (see references 2, 3). Deep tissue injury can be extremely perilous, as it can evolve undetected until a significant destruction of the tissue has occurred. Presently, pressure ulcers are detected by visual inspection of the skin (see reference 45), which often belies existing extensive damage to deeper tissue (see reference 11).

At the present time, techniques employed to prevent ulcer formation include frequent repositioning (see reference 12) as well as the use of specialized cushions and mattresses that provide either static or dynamic pressure relief of the tissues at risk (see reference 22, 46). Recognizing the absence of a significant reduction in the incidence of pressure ulcers (see references 10, 15, 16, 30, 42, 49, 50, 54), new preventative interventions are needed, especially for DTI.

### SUMMARY OF THE INVENTION

In one aspect, there is provided a method for mitigating or preventing formation of pressure ulcers in a patient by transmitting an electrical stimulus to a skin portion of a patient sufficient to effect contraction of a muscle, wherein the method includes a first mode of operation and a second mode of operation, comprising: over a period of at least about one hour, continuously switching between the first mode of operation and the second mode of operation, wherein, for each instance of the first mode of operation, the first mode of operation lasts a respective predetermined stimulus time duration, and a respective operative electrical stimulus, sufficient to effect contraction of a muscle, is transmitted to the skin portion, thereby effecting contraction of the muscle, during the entire, or substantially the entire, respective predetermined stimulus time duration. and wherein, for each instance of the second mode of operation, the second mode of operation lasts a respective predetermined relaxation time duration, and the muscle is relaxed during the entire, or substantially the entire, respective predetermined relaxation time duration.

In another aspect, there is provided a method for mitigating or preventing formation of pressure ulcers in a patient, comprising over a period of at least about one hour, transmitting a plurality of intermittent transmissions of an electrical stimulus, each one of the plurality of intermittent transmissions sufficient to effect contraction of a muscle, to a skin portion of the patient, thereby effecting contraction of the muscle wherein a respective time interval is provided between each pair of successive intermittent transmissions of the plurality of intermittent transmissions, such that there is a plurality of respective time intervals, and wherein the muscle is relaxed during the entirety, or the substantial entirety, of each one of the plurality of respective time intervals.

In another aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions is transmitted over a period of at least about one hour, and wherein a respective time interval is provided between each pair of successive intermittent transmissions of the plurality of intermittent transmissions, such that there is a plurality of respective time intervals, and wherein the muscle is relaxed during the entirety, or the substantial entirety, of each one of the plurality of respective time intervals.

In another aspect, there is provided a method for mitigating or preventing formation of pressure ulcers in a patient by transmitting an electrical stimulus to a skin portion of a patient sufficient to effect contraction of a muscle, wherein the method includes a first mode of operation and a second mode of operation, comprising continuously switching between the first mode of operation and the second mode of operation, wherein for each instance of the first mode of operation, the first mode of operation lasts a respective predetermined stimulus time duration, and a respective operative electrical stimulus, sufficient to effect contraction of a muscle, is transmitted to the skin portion, thereby effecting contraction of the muscle, during the entire, or substantially the entire, respective predetermined stimulus time duration and wherein, for each instance of the second mode of operation, the second mode of operation lasts a respective predetermined relaxation time duration, and the muscle is relaxed during the entire, or substantially the entire, respective predetermined relaxation time duration, and wherein the respective predetermined relaxation time duration of each instance of the second mode of operation is at least five (5) minutes.

In another aspect, there is provided a method for mitigating or preventing formation of pressure ulcers in a patient, comprising transmitting a plurality of intermittent transmissions of an electrical stimulus, each one of the plurality of intermittent transmissions sufficient to effect contraction of a muscle, to a skin portion of a patient, thereby effecting contraction of the muscle, wherein a respective time interval is provided between each pair of successive intermittent transmissions of the plurality of intermittent transmissions, such that there is a plurality of respective time intervals, and wherein the muscle is relaxed during the entirety, or the substantial entirety, of each one of the plurality of respective time intervals, and wherein each one of the plurality of respective time intervals is at least five (5) minutes.

In another aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein a respective time interval is provided between each pair of successive intermittent transmissions of the plurality of intermittent transmissions, such that there is a plurality of respective time intervals, and wherein the muscle is relaxed during the entirety, or the substantial entirety, of each one of the plurality of respective time intervals, and wherein each one of the plurality of respective time intervals is at least five (5) minutes.

In another aspect, there is provided a method for mitigating or preventing formation of pressure ulcers in a patient by transmitting an electrical stimulus to a skin portion of a patient sufficient to effect contraction of a muscle, wherein the method includes a first mode of operation and a second mode of operation, comprising over a period of at least about one hour, continuously switching between the first mode of operation and the second mode of operation, wherein, for each instance of the first mode of operation, the first mode of operation lasts a respective predetermined stimulus time duration, and a respective operative electrical stimulus, sufficient to effect contraction of a muscle, is transmitted to a skin portion by an electrode, in contact with the skin portion, thereby effecting contraction of the muscle, during the entire, or substantially the entire, respective predetermined stimulus time duration, and wherein, for each instance of the second mode of operation, the second mode of operation lasts a respective predetermined relaxation time duration, and no electrical stimulus, or substantially no electrical stimulus, is transmitted to the skin portion by the electrode during the entire, or substantially the entire, respective predetermined relaxation time duration.

In another aspect, there is provided a method for mitigating or preventing formation of pressure ulcers in a patient, comprising over a period of at least about one hour, and by way of an electrode in contact with a skin portion of the patient, transmitting a plurality of intermittent transmissions of an electrical stimulus, each one of the plurality of intermittent transmissions sufficient to effect contraction of a muscle, to the skin portion of the patient, thereby effecting contraction of the muscle, wherein a respective time interval is provided between each pair of successive intermittent transmissions of the plurality of intermittent transmissions, such that there is a plurality of respective time intervals, and wherein no electrical stimulus, or substantially no electrical stimulus, is transmitted to the skin portion by the electrode during the entirety, or the substantial entirety, of each one of the plurality of respective time intervals.

In another aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions is transmitted over a period of at least about one hour by an electrode in contact with the skin portion, and wherein a respective time interval is provided between each pair of successive intermittent transmissions of the plurality of intermittent transmissions, such that there is a plurality of respective time intervals, and wherein no electrical stimulus, or substantially no electrical stimulus, is transmitted to the skin portion by the electrode during the entirety, or the substantial entirety, of each one of the plurality of respective time intervals.

In another aspect, there is provided a method for mitigating or preventing formation of pressure ulcers in a patient by transmitting an electrical stimulus to a skin portion of a patient sufficient to effect contraction of a muscle, wherein the method includes a first mode of operation and a second mode of operation, comprising continuously switching between the first mode of operation and the second mode of operation, wherein, for each instance of the first mode of operation, the first mode of operation lasts a respective predetermined stimulus time duration, and a respective operative electrical stimulus, sufficient to effect contraction of a muscle, is transmitted to the skin portion by an electrode, in contact with the skin portion, thereby effecting contraction of the muscle, during the entire, or substantially the entire, respective predetermined stimulus time duration, and wherein, for each instance of the second mode of operation, the second mode of operation lasts a respective predetermined relaxation time duration, and the muscle is relaxed during the entire, or substantially the entire, respective predetermined relaxation time duration, and wherein the respective predetermined relaxation time duration of each instance of the second mode of operation is at least five (5) minutes.

In another aspect, there is provided a method for mitigating or preventing formation of pressure ulcers in a patient, comprising transmitting a plurality of intermittent transmissions of an electrical stimulus with an electrode in contact with a skin portion of the patient, each one of the plurality of intermittent transmissions sufficient to effect contraction of a muscle, to the skin portion, thereby effecting contraction of the muscle, wherein a respective time interval is provided between each pair of successive intermittent transmissions of the plurality of intermittent transmissions, such that there is a plurality of respective time intervals, and wherein no electrical stimulus, or substantially no electrical stimulus, is transmitted to the skin portion by the electrode during the entirety, or the substantial entirety, of each one of the plurality of respective time intervals, and wherein each one of the plurality of respective time intervals is at least five (5) minutes.

In another aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions is transmitted by an electrode in contact with the skin portion, and wherein a respective time interval is provided between each pair of successive intermittent transmissions of the plurality of intermittent transmissions, such that there is a plurality of respective time intervals, and wherein no electrical stimulus, or substantially no electrical stimulus, is transmitted to the skin portion by the electrode during the entirety, or the substantial entirety, of each one of the plurality of respective time intervals, and wherein each one of the plurality of respective time intervals is at least five (5) minutes.

In another aspect, there is provided a method for mitigating or preventing formation of pressure ulcers in a patient comprising effecting a treatment, wherein the treatment is intermittently transmitting an electrical stimulus to a skin portion of a patient sufficient to effect contraction of a muscle, and thereby effecting contraction of the muscle, wherein the muscle being contracted is not pre-conditioned immediately prior to the treatment

In another aspect, there is provided use of an intermittent electrical stimulus being transmitted to a skin portion of a patient and sufficient to effect contraction of a muscle, and thereby effecting contraction of the muscle to effect a treatment for mitigating or preventing formation of pressure ulcers in the patient, wherein the muscle being contracted is not pre-conditioned immediately prior to the treatment.

In another aspect, there is provided a method for mitigating or preventing formation of pressure ulcers in a patient comprising intermittently transmitting an electrical stimulus to a skin portion of a patient sufficient to effect contraction of a muscle, and thereby effecting contraction of the muscle, wherein the electrical stimulus being transmitted is insufficient to effect lifting of the patient or movement of the limbs.

In another aspect, there is provided use of an intermittent electrical stimulus being transmitted to a skin portion of a patient and sufficient to effect contraction of a muscle, and thereby effecting contraction of the muscle in order to mitigate or prevent formation of pressure ulcers in the patient, wherein the electrical stimulus being transmitted is insufficient to effect lifting of the patient or movement of the limbs.

In another aspect, there is provided, a method for mitigating or preventing formation of pressure ulcers in a patient comprising intermittently transmitting an electrical stimulus to a skin portion of a patient sufficient to effect contraction of a muscle, and thereby effecting contraction of the muscle, wherein the patient is disposed in a supine position or in a recumbence position when the skin portion is receiving the electrical stimulus.

In another aspect, there is provided Use of an electrical stimulus being transmitted to a skin portion of a patient sufficient to effect contraction of a muscle, and thereby effecting contraction of the muscle in order to mitigate or prevent formation of pressure ulcers in the patient, wherein the patient is disposed in a supine position or in a recumbence position when the skin portion is receiving the electrical stimulus.

### BRIEF DESCRIPTION OF DRAWINGS

The invention will better be understood when consideration is given to the following detailed description thereof. Such description makes reference to the annexed drawings wherein:

FIG. 1a is a top schematic view of the experimental set-up using a rat as the experimental subject, illustrating how constant pressure was applied to the quadriceps muscle of the right hind limb of a rat;

FIG. 1b is a graph illustrating a 50-minute record of the force applied to the quadriceps muscle of a rat; the sharp increases in force corresponding to the contraction of muscle due to intermittent electrical stimulation (IES);

FIGS. 1c and 1d are top and side views of experimental set up for the rat experiments, illustrating the locations of the electrode and leads for electrical stimulation (input) and the force transducer for measuring the force (output) generated by the contraction of the muscles are demonstrated, and also is shown the indenter that was used to apply external force to the body in order to load the muscle and surrounding tissue to levels that mimic the loading levels experienced by individuals sitting in a wheelchair or lying down in a bed;

FIG. 1e illustrates the force generated by contraction of muscle in response to the intermittent electrical stimulation treatment applied to quadriceps muscles in the rats during two hours of treatment having relaxation periods of: (i) 5 minutes, and (ii) 10 minutes;

FIG. 2 is magnetic resonance imaging scans of one animal, illustrating sequential T2-weighted spin echo magnetic resonance images (MRI's) of a rat's thigh ranging from the rostral extent of the quadriceps muscle (a) to its caudal end (j), obtained 24 hours after the application of external pressure, with approximate placement of indenter indicated in slice (f).

FIG. 3a is a T2-weighted spin-echo magnetic resonance image of rat hind limbs 24 hours after the application of pressure; FIG. 3b are magnetic resonance images of the quadriceps muscle. FIG. 3c are magnetic resonance images of the quadriceps muscle in both hind limbs, the signal intensity of pixels within the region of the left and right quadriceps muscles; was obtained, and the signal intensity from pixels in the experimental limb was compared to the average+2*standard deviation intensity of those in the contralateral limb, pixels with higher intensity in the experimental limb were marked with red and considered to contain increased water content, pixels with higher intensity than threshold in the contralateral limb were marked with blue as a control; Cnt Grp=control group; Exp Grp 1-3=experimental groups 1-3; Contra Cnt Grp=contralateral control group;

FIG. 4 are images of sample hematoxylin and eosin-stained cross sections from different animals in each group; Cnt Grp=control group; Exp Grp 1-3=experimental groups 1-3;

FIG. 5 is summary of magnetic resonance imaging and histology results; Left Axis: individual data points (filled circles) and mean±SD (filled triangles) representing the percent of muscle volume with increased water content (edema) in the quadriceps muscle in all rat groups; right axis: Individual data points (empty circles) and mean±SD (empty triangles) representing the necrosis score from the quadriceps muscle in all rat groups, and scoring for quantifying muscle necrosis (per 4.9 mm² of muscle area) is: 0=no necrosis in region analyzed; 1=0-10% of region analyzed exhibited necrosis; 2=10-25%; 3=25-50%; 4>50%. (*represents statistically significant difference, PCT.05);

FIG. 6 illustrates changes in levels of oxygenation and surface pressure due to loading and IES (a) quantitative T2* imaging following 6, 30-sec bouts of electrical stimulation applied to medial gastrocnemius, persistent regional increases in blood oxygenation were seen with IES; (b) quantitative T2* imaging of the gluteus maximus muscles under different conditions, a persistent decrease in blood oxygenation was seen when the muscles were loaded, a persistent increase was obtained with IES; (c) surface-skin interface pressure map of the gluteus muscles under different conditions, and highest point of pressure with loading was observed around the sacrum (arrows); with IES, pressure became more evenly distributed, eliminating the previous concentrations of high pressure; ROI 1-4=region of interest 1-4; pre-stim=before electrical stimulation; post 1-6=after 1-6 bouts of electrical stimulation;

FIG. 7 illustrates the experimental set-up for human volunteers;

FIG. 8 shows an MRI of the left and right gluteus muscles demonstrating the changes in muscle shape during contractions induced by electrical stimulation (top) and also shows the redistribution of surface pressure (middle) and the increase in tissue oxygenation (bottom) during electrical stimulation;

FIG. 9 shows the locations of the electrodes for delivering the intermittent electrical stimulation treatment in the various positions in which a patient would be disposed;

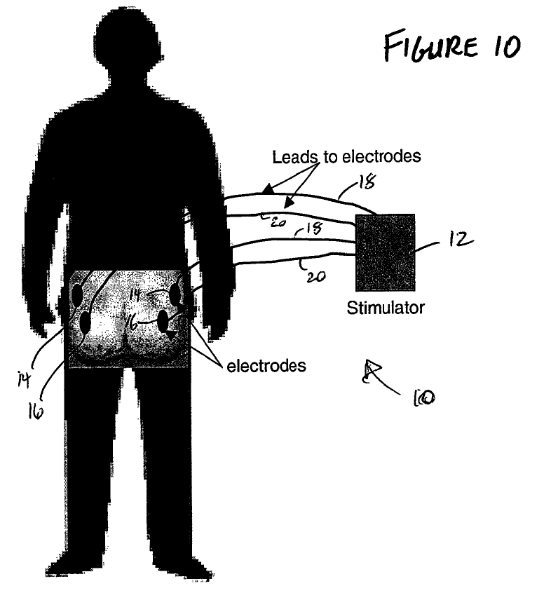

FIG. 10 is a schematic illustration of a system for effecting the mitigation or prevention of formation of pressure ulcers by transmitting an electrical stimulus to the skin of a human patient;

FIGS. 11a, b, c, and d illustrate examples of intermittent electrical stimulation pattern;

FIG. 12 illustrates quantified pressure mat sensor readings showing reductions in surface pressure around the ischial tuberosities induced by FES;

FIG. 13 illustrates magnetic resonance images showing changes in muscle shape during IES; and

FIG. 14 illustrates sustained increases in tissue oxygenation produced by contractions generated during the ON period of IES.

### DETAILED DESCRIPTION OF PREFERRED EMBODIMENTS

Unless otherwise indicated, all numbers expressing quantities of ingredients, properties such as distance, operating conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain errors necessarily resulting from the standard deviation found in their respective testing measurements.

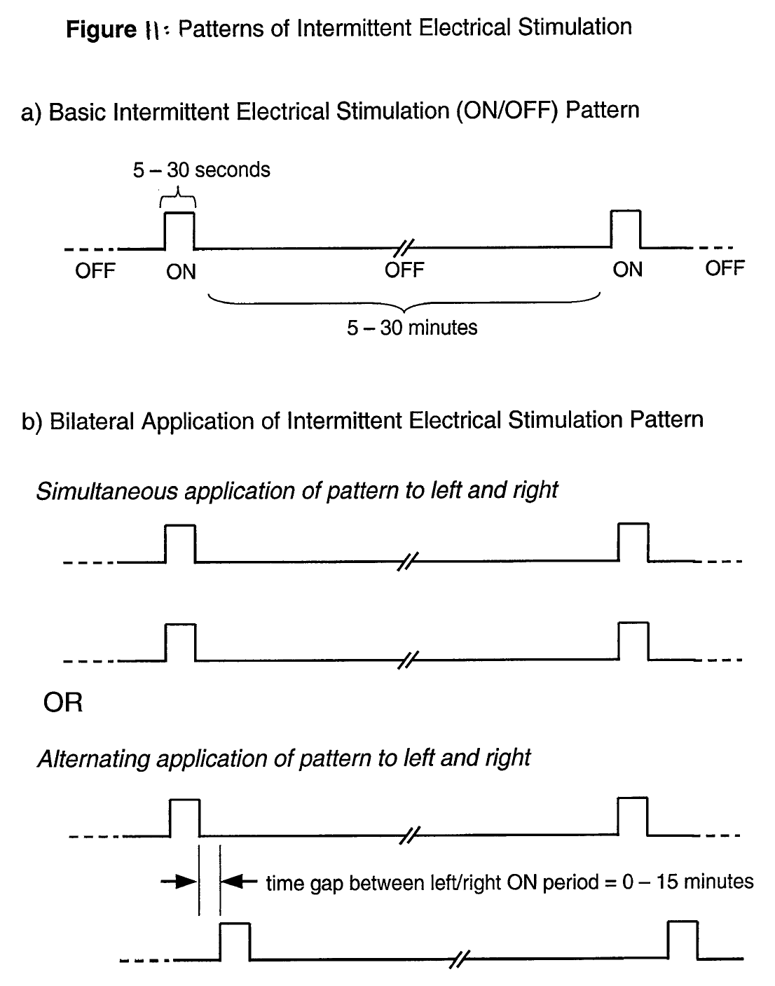

There is provided a method for mitigating or preventing formation of pressure ulcers in a patient by transmitting an electrical stimulus to a skin portion of a patient sufficient to effect contraction of a muscle. There is also provided a use of an electrical stimulation of a skin portion of a patient sufficient to effect contraction of a muscle of a patient for mitigating or preventing formation of pressure ulcers in the patient. An example of a system for effecting this use or method is illustrated in FIG. 10. The system includes a stimulator 12 which is electrically coupled to each of an anode 14 and a cathode 16 with a respective one of the leads 18, 20. Each one of the anode 14 and the cathode 16 is in contact with a skin portion of the patient. For example, each one of the anode 14 and the cathode 16 may be embedded in clothing worn by the patient. For example, skin burns may be mitigated by measuring the impedance of the electrode-tissue interface and limiting the amount of voltage that can be applied by the stimulator across the interface. For example, the stimulator is battery operated.

As mentioned above, a pressure ulcer is any lesion caused by unrelieved pressure resulting in damage of underlying tissue, involving any one of or any combination of skin, fat, fascia, muscle, or bone, and a pressure ulcer develops following a prolonged period of compression of the tissue between a bony prominence and a surface.

For example, the pressure ulcer is a deep tissue injury, as explained above.

For example, with respect to the transmission of the electrical stimulus, the electrode stimulus is transmitted to the skin portion by an electrode in contact with the skin portion. For example, the electrical stimulus is an electrical signal. For example, the electrical signal is a discrete signal (eg. pulsatile waveform), a continuous signal (eg. sustained sinusoidal waveform, rectangular waveform), or a combination of a discrete signal and a continuous signal. For example, with respect to the electrical signal, the electrical signal includes a characteristic frequency of 20 Hz to 60 Hz. For example the electrical signal includes a characteristic frequency of 40 Hz.

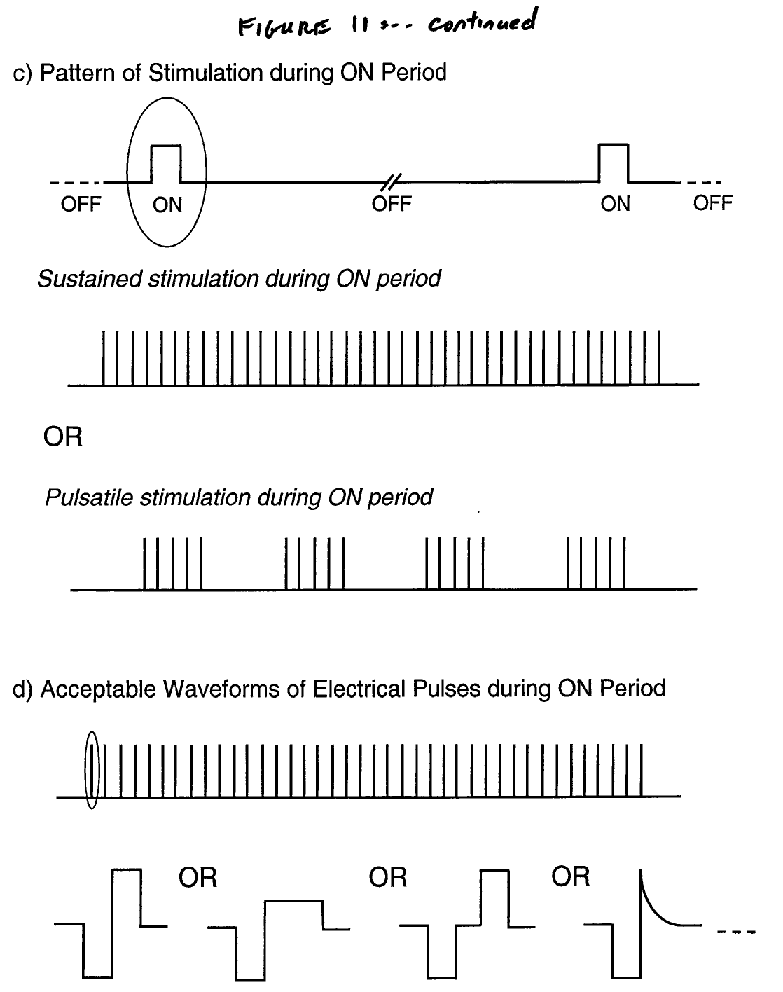

FIGS. 11 a, b, c, d illustrate examples of intermittent electrical stimulation patterns. FIG. 11a illustrates a basic intermittent electrical stimulation pattern. FIG. 11b illustrates an example of intermittent electrical stimulation pattern for bilateral stimulation. Bilateral stimulation refers to the application of the basic intermittent electrical stimulation pattern, which includes an ON mode (also described below as a “first mode of operation”) and an OFF mode (also described below as a “second mode of operation”), to the muscles on both sides of the body (eg. left and right gluteus maximus muscles). The ON mode of pattern to the muscles on both sides of the body can occur simultaneously (i.e. stimulation of the muscles take place at the same time). Or, the ON mode of stimulation to the muscles on both sides of the body can be staggered. For example, the ON mode to one side can occur at the end of the ON mode to the other side, or up to 15 minutes from the end of the ON mode to the other side. FIG. 11c illustrates continuous (or “sustained”) and pulsatile applications during the ON mode of the pattern. FIG. 11d illustrates the general waveforms of each stimulus pulse.

For example, with respect to the skin portion, the skin portion is a skin portion underneath which lies the nerve controlling the contractions of a muscle, and which is supported by a support surface. For example, the skin portion, to which the electrical signal is transmitted, is a skin portion proximate to the tissue for which the pressure ulcer is intended to be mitigated. For example, the force exerted by the support surface, as the support surface is supporting the skin portion, effects compression of tissue disposed between the skin portion and a bony prominence. For example, the skin portion is provided on the buttocks of the patient. In this respect, for example, the support surface is a seating surface.

For example, with respect to the effect of the transmission of the electrical signal to the skin portion, the electrical stimulation effects contraction of the muscle, thereby reshaping the form of the muscle and redistributing pressure away from the tissue for which the pressure ulcer is intended to be mitigated, and also increasing oxygenation of tissue which may have suffered from ischemia.

In one aspect, there is provided a method for mitigating or preventing formation of pressure ulcers in a patient comprising effecting a treatment, wherein the treatment is intermittently transmitting an electrical stimulus to a skin portion of a patient sufficient to effect contraction of a muscle, and thereby effecting contraction of the muscle, wherein the muscle being contracted is not pre-conditioned immediately prior to the treatment. In a related aspect, there is provided a use of an intermittent electrical stimulus being transmitted to a skin portion of a patient and sufficient to effect contraction of a muscle, and thereby effecting contraction of the muscle and effecting a treatment for mitigating or preventing formation of pressure ulcers in the patient, wherein the muscle being contracted is not pre-conditioned immediately prior to the treatment. Pre-conditioning of the muscles means applying electrical stimulation to the muscle for a period of time (for example, several hours) each day, for a number of days immediately prior to the treatment (for example, several months, and usually at least three months). During the initial phases of the pre-conditioning period, stimulation is applied for a minimum of one (1) hour per day. This period is increased to several hours, with some people having the stimulation applied for 12 hours or more. The purpose of the pre-conditioning is for the purpose of increasing muscle mass and improving muscle endurance (fatigue resistance). For example, in the 60 day period immediately prior to the treatment, the mass of the muscle intended to be contracted during the treatment does not significantly increase. In this respect, for example, during the 60 day period immediately prior to the treatment, the mass of the muscle intended to be contracted during the treatment increases less than 5%. As a further example, during the 60 day period immediately prior to the treatment, the endurance of the muscle intended to be contracted during the treatment does not significantly increase. In this respect, for example, during the 60 day period immediately prior to the treatment, the endurance of the muscle intended to be contract increases less than 5%. In this context, changes in endurance are measured in accordance with the endurance (fatigue) test provided in R. B. Stein, T. Gordon, J. Jefferson, A. Sharfenberger, J. F. Yang, J. T. de Zepetnek, and M. Belanger (1992), “Optimal stimulation of paralyzed muscle after human spinal cord injury”. Journal of Applied Physiology 72(4):1393-400, which is incorporated in its entirety herein by reference, and more particularly, is provided in the last paragraph on page 1394 of that article, at the passage beginning with: “Fatigue was measured by . . . ”.

In another aspect, there is provided a method for mitigating or preventing formation of pressure ulcers in a patient comprising intermittently transmitting an electrical stimulus to a skin portion of a patient sufficient to effect contraction of a muscle, and thereby effecting contraction of the muscle, wherein the electrical stimulus being transmitted is insufficient to effect lifting of the patient or movement of the limbs. In this respect, the electrical stimulus being transmitted is configured to effect minimal joint movement. In a related aspect, there is provided a use of an intermittent electrical stimulus being transmitted to a skin portion of a patient and sufficient to effect contraction of a muscle, and thereby effecting contraction of the muscle in order to mitigate or prevent formation of pressure ulcers in the patient, wherein the electrical stimulus being transmitted is insufficient to effect lifting of the patient or movement of the limbs. For example, with respect to the lifting, the lifting is of at least a portion of the patient's body relative to a support surface supporting the patient (eg. the buttocks region lifting above the wheelchair seat). For example, the electrical stimulus being transmitted effects isometric contraction of the muscle. For example, the electrical stimulus effects a change in the angle between two bones defining each and every joint of the patient by less than 10 degrees (ie. upon application of the electrical stimulus, none of the joints of the patient change by 10 degrees or more). For example, when the patient is in the sitting position, stimulation of the gluteus maximus would cause an isometric contraction of the muscle with less than 10 degrees change in the hip joint angle. For example, when the patient is in the supine position, stimulation of the gluteus maximus would cause an isometric contraction of the muscles with less than 10 degrees change in the hip joint angle and lumbar spine. As a further example of when the patient is in the supine position, stimulation of trapezius would cause an isomeric contraction of the muscle with less than 10 degrees change in the shoulder joint angle. As a further example of when the patient is in the supine position, stimulation of the muscles of the back of the head would cause an isometric contraction of the muscles with less than 10 degrees change in the angle of the neck relative to the head. For example, when the patient is in the lateral recumbence position, stimulation of the deltoid would cause an isometric contraction of the muscle with less than 10 degrees change in the shoulder angle. As a further example of when the patient is in the lateral recumbence position, stimulation of the gluteus medius muscle would cause an isometric contraction of the muscle with less than 10 degrees change in the hip angles. As a further example of when the patient is in the lateral recumbence position, stimulation of tensor fascia latae would cause an isometric contraction of the muscle with less than 10 degrees change in the hip joint angle or knee angle.

In another aspect, there is provided a method for mitigating or preventing formation of pressure ulcers in a patient comprising intermittently transmitting an electrical stimulus to a skin portion of a patient sufficient to effect contraction of a muscle, and thereby effecting contraction of the muscle, wherein the patient is disposed in a supine position or in a recumbence position when the skin portion is receiving the electrical stimulus. In a related aspect, there is provided a use of an electrical stimulus being transmitted to a skin portion of a patient sufficient to effect contraction of a muscle, and thereby effecting contraction of the muscle in order to mitigate or prevent formation of pressure ulcers in the patient, wherein the patient is disposed in a supine position or in a recumbence position when the skin portion is receiving the electrical stimulus. For example, when the patient is disposed in a supine position, the muscle is any one of a gluteus muscle, a muscle at least partially surrounding the shoulder blades of the patient, or a muscle disposed in proximity to the back of the head of the patent. For example, when the patient is disposed in a lateral recumbence position, the muscle is a muscle of the hip (eg, gluteus medius and tensor fascia latae muscles) or the side muscles surrounding the shoulder (eg, deltoid muscle).

In another aspect, there is provided a method for mitigating or preventing formation of pressure ulcers in a patient, including a first mode of operation and a second mode of operation. The method comprises, over a period of at least one hour, continuously switching between the first mode of operation and the second mode of operation. For each instance of the first mode of operation, the first mode of operation lasts a respective predetermined stimulus time duration, and a respective operative electrical stimulus, sufficient to effect contraction of a muscle, is transmitted to a skin portion of a patient, thereby effecting contraction of the muscle, during the entire, or substantially the entire, respective predetermined stimulus time duration. For each instance of the second mode of operation, the second mode of operation lasts a respective predetermined relaxation time duration, and the muscle is relaxed during the entire, or substantially the entire, respective predetermined relaxation time duration. The switching from an instance of the first mode of operation to an instance of the second mode of operation is effected when the respective predetermined stimulus time duration of the respective instance of the first mode of operation is completed. As well, switching from an instance of the second mode of operation to an instance of the first mode of operation is effected when the respective predetermined relaxation time duration of the respective instance of the second mode of operation is completed. For example, the respective predetermined stimulus time duration of each instance of the first mode of operation is at least seven (7) seconds. As a further example, the respective predetermined stimulus time duration of each instance of the first mode of operation is greater than seven (7) seconds and less than (30) seconds. As a further example, the respective predetermined stimulus time duration of each instance of the first mode of operation is ten (10) seconds. For example, the respective predetermined relaxation time duration of each instance of the second mode of operation is at least five (5) minutes. As a further example, with respect to the respective predetermined relaxation time duration, the respective predetermined relaxation time duration of each instance of the second mode of operation is at least ten (10) minutes. As a further example, with respect to the respective predetermined relaxation time duration, the respective predetermined relaxation time duration of each instance of the second mode of operation is ten (10) minutes. For example, the continuous switching between the first mode of operation and the second mode of operation is effected over a period of at least two hours.

In a related aspect, there is provided a method of mitigating or preventing formation of pressure ulcers in a patient, comprising, over a period of at least one hour, transmitting a plurality of intermittent transmissions of an electrical stimulus, each one of the plurality of intermittent transmissions being sufficient to effect contraction of a muscle, to a skin portion of a patient, thereby effecting contraction of the muscle. A respective time interval is provided between each pair of successive intermittent transmissions of the plurality of intermittent transmissions of an electrical stimulus, such that there is a plurality of respective time intervals. The muscle is relaxed during the entirety, or the substantial entirety, of each one of the plurality of respective time intervals. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds. For example, each one of the plurality of respective time intervals is at least five (5) minutes. As a further example, each one of the plurality of respective time intervals is at least ten (10) minutes. As a further example, each one of the plurality of respective time intervals is ten (10) minutes. For example, the plurality of intermittent transmissions is effected over a period of at least two hours.

In a further related aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions of an operative electrical stimulus is transmitted over a period of at least one hour. A respective time interval is provided between each pair of successive intermittent transmissions of the plurality of intermittent transmissions of an electrical stimulus, such that there is a plurality of respective time intervals. The muscle is relaxed during the entirety, or the substantial entirety, of each one of the plurality of respective time intervals. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds. For example, each one of the plurality of respective time intervals is at least five (5) minutes. As a further example, each one of the plurality of respective time intervals is at least ten (10) minutes. As a further example, each one of the plurality of respective time intervals is ten (10) minutes. For example, the plurality of intermittent transmissions is effected over a period of at least two hours.

In another aspect, there is provided a method for mitigating or preventing formation of pressure ulcers in a patient, including a first mode of operation and a second mode of operation. The method comprises continuously switching between the first mode operation and the second mode of operation. For each instance of the first mode of operation, the first mode of operation lasts a respective predetermined stimulus time duration, and a respective operative electrical stimulus, sufficient to effect contraction of a muscle, is transmitted to a skin portion of a patient, thereby effecting contraction of the muscle, during the entire, or substantially the entire, respective predetermined stimulus time duration. For each instance of the second mode of operation, the second mode of operation lasts a respective predetermined relaxation time duration, and the muscle is relaxed during the entire, or substantially the entire, respective predetermined relaxation time duration. The switching from an instance of the first mode of operation to an instance of the second mode of operation is effected when the respective predetermined stimulus time duration of the respective instance of the first mode of operation is completed. As well, switching from an instance of the second mode of operation to an instance of the first mode of operation is effected when the respective predetermined relaxation time duration of the respective instance of the second mode of operation is completed. The respective predetermined relaxation time duration of each instance of the second mode of operation is at least five (5) minutes. For example, the respective predetermined relaxation time duration of each instance of the second mode of operation is at least ten (10) minutes. For example, the respective predetermined relaxation time duration of each instance of the second mode of operation is ten (10) minutes. For example, the respective predetermined stimulus time duration of each instance of the first mode of operation is at least seven (7) seconds. As a further example, the respective predetermined stimulus time duration of each instance of the first mode of operation is greater than seven (7) seconds and less than (30) seconds. As a further example, the respective predetermined stimulus time duration of each instance of the first mode of operation is ten (10) seconds.

In a related aspect, there is provided a method of mitigating or preventing formation of pressure ulcers in a patient, comprising transmitting a plurality of intermittent transmissions of an electrical stimulus, sufficient to effect contraction of a muscle, to a skin portion of a patient, thereby effecting contraction of the muscle. A respective time interval is provided between each pair of successive intermittent transmissions of the plurality of intermittent transmissions of an electrical stimulus such that there is a plurality of respective time intervals. The muscle is relaxed during the entirety, or the substantial entirety, of each one of the plurality of respective time intervals. Each one of the plurality of respective time intervals is at least five (5) minutes. For example, each one of the plurality of respective time intervals is at least ten (10) minutes. For example, each one of the plurality of respective time intervals is ten (10) minutes. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds.

In a further related aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient. A respective time interval is provided between each pair of successive intermittent transmissions of the plurality of intermittent transmissions of an electrical stimulus such that there is a plurality of respective time intervals. The muscle is relaxed during the entirety or the substantial entirety of each one of the plurality of respective time intervals. Each one of the plurality of respective time intervals is at least five (5) minutes. As a further example, each one of the plurality of respective time intervals is at least ten (10) minutes. As a further example, each one of the plurality of respective time intervals is ten (10) minutes. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds.

The term “relaxed”, when used above to describe the muscle, mean that the muscle is not being induced to contract by an electrical stimulus.

For example, with respect to the second mode of operation, the respective relaxation time duration of each instance of the second mode of operation is provided to provide sufficient rest for the muscle in between the transmissions of the electrical stimuli during successive instances of the first mode of operation. Without providing this relaxation time duration, the muscle may become fatigued much earlier during the method or use of the intermittent transmissions, and require an unacceptable early termination of the treatment or the use of the intermittent transmissions.

In another aspect, there is provided a method for mitigating or preventing formation of pressure ulcers in a patient, including a first mode of operation and a second mode of operation. The method comprises, over a period of at least one hour, continuously switching between the first mode of operation and the second mode of operation. For each instance of the first mode of operation, the first mode of operation lasts a respective predetermined stimulus time duration, and a respective operative electrical stimulus, sufficient to effect contraction of a muscle, is transmitted to a skin portion of a patient by an electrode, thereby effecting contraction of the muscle, during the entire, or substantially the entire, respective predetermined stimulus time duration, wherein the electrode is in contact with the skin portion. For each instance of the second mode of operation, the second mode of operation lasts a respective predetermined relaxation time duration, and no electrical stimulus, or substantially no electrical stimulus, is transmitted to the skin portion by the electrode during the entire, or substantially the entire, respective predetermined relaxation time duration. The switching from an instance of the first mode of operation to an instance of the second mode of operation is effected when the respective predetermined stimulus time duration of the respective instance of the first mode of operation is completed. As well, switching from an instance of the second mode of operation to an instance of the first mode of operation is effected when the respective predetermined relaxation time duration of the respective instance of the second mode of operation is completed. For example, the respective predetermined stimulus time duration of each instance of the first mode of operation is at least seven (7) seconds. As a further example, the respective predetermined stimulus time duration of each instance of the first mode of operation is greater than seven (7) seconds and less than (30) seconds. As a further example, the respective predetermined stimulus time duration of each instance of the first mode of operation is ten (10) seconds. For example, the respective predetermined relaxation time duration of each instance of the second mode of operation is at least five (5) minutes. As a further example, with respect to the respective predetermined relaxation time duration, the respective predetermined relaxation time duration of each instance of the second mode of operation is at least ten (10) minutes. As a further example, with respect to the respective predetermined relaxation time duration, the respective predetermined relaxation time duration of each instance of the second mode of operation is ten (10) minutes. For example, the continuous switching between the first mode of operation and the second mode of operation is effected over a period of at least two hours.

In a related aspect, there is provided a method of mitigating or preventing formation of pressure ulcers in a patient, comprising, over a period of at least one hour, and by way of an electrode in contact with a skin portion of a patient, transmitting a plurality of intermittent transmissions of an electrical stimulus, sufficient to effect contraction of a muscle, to the skin portion of a patient, thereby effecting contraction of the muscle. A respective time interval is provided between each pair of successive intermittent transmissions of the plurality of intermittent transmissions of an electrical stimulus such that there is a plurality of respective time intervals. No electrical stimulus, or substantially no electrical stimulus, is transmitted to the skin portion by the electrode during the entirety, or the substantial entirety, of each one of the plurality of respective time intervals. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds. For example, each one of the plurality of respective time intervals is at least five (5) minutes. As a further example, each one of the plurality of respective time intervals is at least ten (10) minutes. As a further example, each one of the plurality of respective time intervals is ten (10) minutes. For example, the plurality of intermittent transmissions is effected over a period of at least two hours.

In a further related aspect, there is provided use of a plurality of intermittent transmissions of an operative electrical stimulus, sufficient to effect contraction of a muscle, to a skin portion of the patient, thereby effecting contraction of the muscle, for mitigating or preventing formation of pressure ulcers in the patient, wherein the plurality of intermittent transmissions of an operative electrical stimulus is transmitted over a period of at least one hour by an electrode in contact with the skin portion. A respective time interval is provided between each pair of successive intermittent transmissions of the plurality of intermittent transmissions of an electrical stimulus such that there is a plurality of respective time intervals. No electrical stimulus, or substantially no electrical stimulus, is transmitted to the skin portion by the electrode during the entirety, or the substantial entirety, of each one of the plurality of respective time intervals. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds. For example, each one of the plurality of respective time intervals is at least five (5) minutes. As a further example, each one of the plurality of respective time intervals is at least ten (10) minutes. As a further example, each one of the plurality of respective time intervals is ten (10) minutes. For example, the plurality of intermittent transmissions is effected over a period of at least two hours.

In another aspect, there is provided a method for mitigating or preventing formation of pressure ulcers in a first mode of operation and a second mode of operation. The method comprises continuously switching between the first mode of operation and the second mode of operation. For each instance of the first mode of operation, the first mode of operation lasts a respective predetermined stimulus time duration, and a respective operative electrical stimulus, sufficient to effect contraction of a muscle, is transmitted to a skin portion of a patient by an electrode, thereby effecting contraction of the muscle, during the entire, or substantially the entire, respective predetermined stimulus time duration, wherein the electrode is in contact with the muscle. For each instance of the second mode of operation, the second mode of operation lasts a respective predetermined relaxation time duration, and no electrical stimulus, or substantially no electrical stimulus, is transmitted to the skin portion by the electrode during the entire, or substantially the entire, respective predetermined relaxation time duration. The switching from an instance of the first mode of operation to an instance of the second mode of operation is effected when the respective predetermined stimulus time duration of the respective instance of the first mode of operation is completed. As well, switching from an instance of the second mode of operation to an instance of the first mode of operation is effected when the respective predetermined relaxation time duration of the respective instance of the second mode of operation is completed. The respective predetermined relaxation time duration of each instance of the second mode of operation is at least five (5) minutes. For example, the respective predetermined relaxation time duration of each instance of the second mode of operation is at least ten (10) minutes. For example, the respective predetermined relaxation time duration of each instance of the second mode of operation is ten (10) minutes. For example, the respective predetermined stimulus time duration of each instance of the first mode of operation is at least seven (7) seconds. As a further example, the respective predetermined stimulus time duration of each instance of the first mode of operation is greater than seven (7) seconds and less than (30) seconds. As a further example, the respective predetermined stimulus time duration of each instance of the first mode of operation is ten (10) seconds.

In a related aspect, there is provided a method of mitigating or preventing formation of pressure ulcers in a patient, comprising, by way of an electrode in contact with a skin portion of a patient, transmitting a plurality of intermittent transmissions of an electrical stimulus, sufficient to effect contraction of a muscle, to the skin portion of a patient, thereby effecting contraction of the muscle. A respective time interval is provided between each pair of successive intermittent transmissions of the plurality of intermittent transmissions of an electrical stimulus such that there is a plurality of respective time intervals. No electrical stimulus, or substantially no electrical stimulus, is transmitted to the skin portion by the electrode during the entirety, or the substantial entirety, of each one of the plurality of respective time intervals. Each one of the plurality of respective time intervals is at least five (5) minutes. For example, each one of the plurality of respective time intervals is at least ten (10) minutes. For example, each one of the plurality of respective time intervals is ten (10) minutes. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds.

In a further related aspect, there is provided use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions of an operative electrical stimulus is transmitted by an electrode in contact with the skin portion. A respective time interval is provided between each pair of successive intermittent transmissions of the plurality of intermittent transmissions of an electrical stimulus such that there is a plurality of respective time intervals. No electrical stimulus, or substantially no electrical stimulus, is transmitted to the skin portion by the electrode during the entirety, or the substantial entirety, of each one of the plurality of respective time intervals. Each one of the plurality of respective time intervals is at least five (5) minutes. As a further example, each one of the plurality of respective time intervals is at least ten (10) minutes. As a further example, each one of the plurality of respective time intervals is ten (10) minutes. For example, each one of the plurality of intermittent transmissions is at least seven (7) seconds. As a further example, each one of the plurality of intermittent transmissions is greater than seven (7) seconds and less than (30) seconds. As a further example, each one of the plurality of intermittent transmissions is ten (10) seconds.

Embodiments of the present invention will be described in further detail with reference to the following non-limitative examples.

### Example No. 1

Experiments have been conducted to investigate the effectiveness of applying intermittent electrical stimulation (IES) to reduce muscle injury due to the presence of persistent external pressure. We hypothesized that the IES-induced muscle contractions would prevent the formation of DTI. These periodically-induced contractions may parallel the effects of voluntary or assisted repositioning, which is the standard method for preventing the formation of DTI. We suggested that the mechanism of action of IES is twofold: 1) IES-induced contractions would reshape the underlying muscle, thereby reducing the high stress levels experienced at the muscle-bone interface, minimizing the amount of damage caused by the mechanical deformation and compression of the tissue. 2) Each contraction would also periodically restore blood flow and increase the oxygenation of the compressed tissue, reducing the amount of damage caused by long periods of ischemia and subsequent reperfusion.

Intermittent electrical stimulation may be a useful medical intervention that allows immobilized individuals to remain seated or supine for prolonged periods of time, reducing the frequency of assisted repositioning, and, most importantly, reducing the development of DTI.

### Overview of Experimental Procedures

To investigate the effectiveness of IES in the prevention of DTI, a series of experiments were conducted in four groups of rats. The Control Group received 2 hours of external load applied to the quadriceps muscle of one hind limb. Experimental Groups 1 and 2 received the load application as well as IES at either 10-minute or 5-minute intervals. Experimental Group 3 received the application of IES at 5-minute intervals but no load application. Deep tissue injury was quantified 24 hours later by in-vivo T₂-weighted magnetic resonance imaging (MRI) and post mortem histological assessment of the extracted quadriceps muscles. The untreated contralateral legs of all animals served as healthy controls (Contralateral Control Group).

To obtain an insight into the mechanisms of action of IES, the effect of IES on tissue oxygenation was measured in two experiments with able-bodied human volunteers. Tissue oxygenation measurements were obtained from an able-bodied volunteer by means of T₂*MRI quantification in muscles in both unloaded and loaded conditions, respectively. A single experiment in an able-bodied volunteer was also performed to measure changes in the surface (bed-buttocks interface) pressure profiles generated by the IES-elicited contractions. All volunteers provided written consent. All experimental protocols were approved by the Animal Care and Welfare Committee and the Health Research Ethics Board at the University of Alberta.

### Effectiveness of IES in Preventing DTI

Pressure Application and Electrical Stimulation Setup

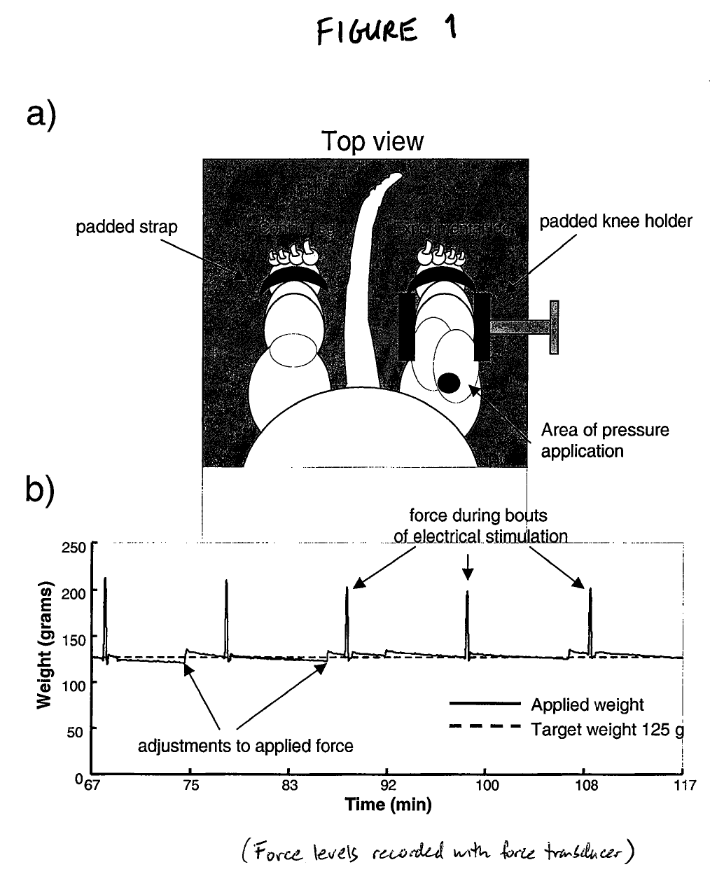

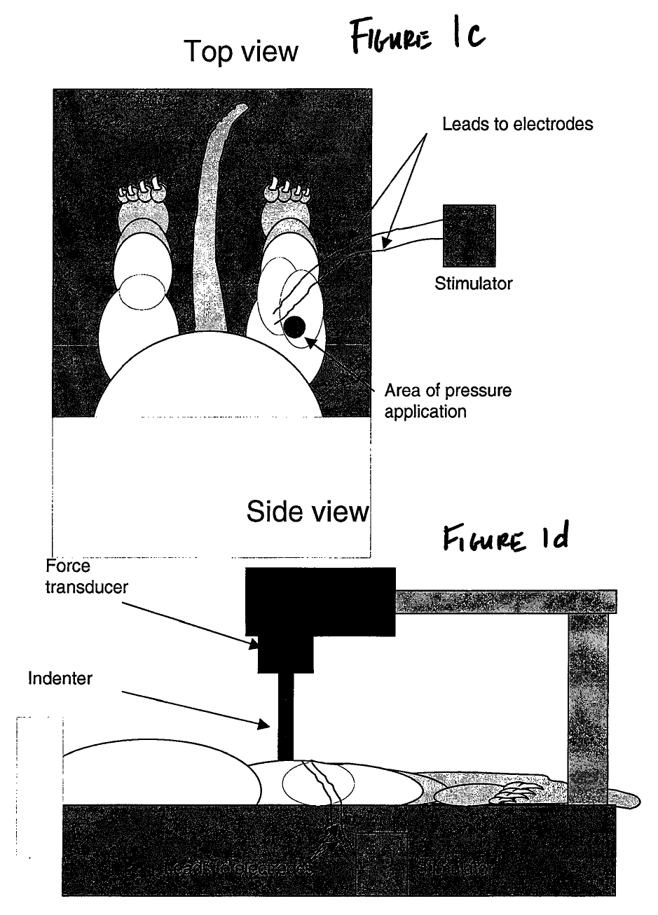

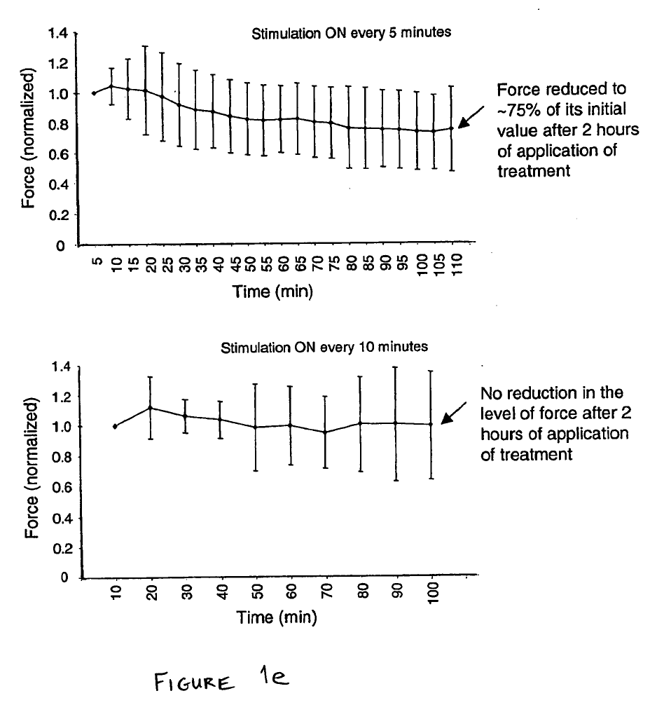

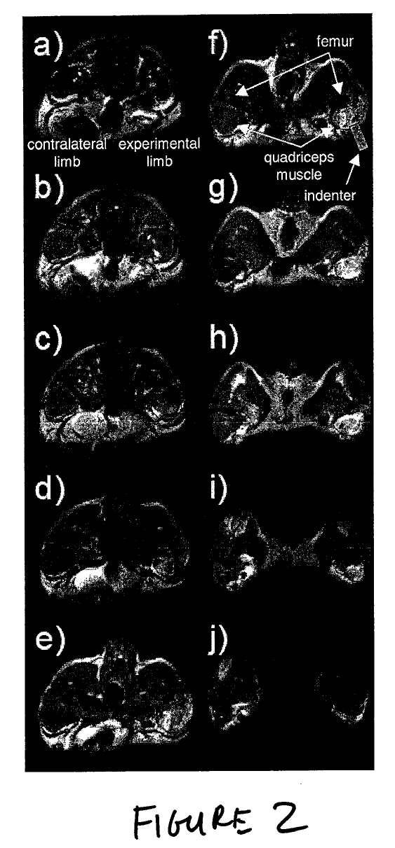

Eighteen adult female, Sprague-Dawley rats (weight=320±36 g) were anesthetized with isoflurane (2-3% isoflurane in 500 ml/min oxygen) and a nerve-cuff was implanted around the femoral nerve of each hind limb. Following implantation the rat was placed on a flat surface with a restraining device (FIG. 1a). Both hind limbs were fully extended and a padded strap was placed around each ankle to tether the legs in place. The knee and upper calf in the experimental leg were also restrained using a padded clamp to prevent any off-sagittal movement of the leg.

Pressure was applied to the quadriceps muscle of the experimental leg using a 3-mm diameter indenter. The contralateral leg served as an internal control. Rats were randomly assigned to 3 groups of 6 animals each (Control Group, Experimental Group 1, Experimental Group 2). Rats in Experimental Group 1 received the application of pressure and simultaneous application of a 10-s stimulus bout (biphasic, charge-balanced, constant current, 10-40 mA, 250 μs, 50 pulses/s) to the femoral nerve of the experimental leg every 10 minutes throughout the duration of pressure application. Rats in Experimental Group 2 received pressure and simultaneous electrical stimulation to the treated leg (10-s bouts) every 5 minutes (see FIG. 1d). Rats in the Control Group received the pressure application but no electrical stimulation. In all animals, pressure was applied for a period of 2 hours. The load applied was normalized to 38% of the body weight of each rat, which is the expected unilateral amount of loading in the buttocks and thighs in seated individuals (see reference 9). Loads were measured with a miniature beam force transducer (Interface, Scottsdale, Ariz., U.S.A.). The force was recorded at a sampling rate of 100 samples/s using a CED Power 1401 A/D board (Cambridge Equipment Design, Cambridge, UK) and SIGNAL 2 software (Cambridge Equipment Design, Cambridge, UK) throughout the duration of the experiment (FIG. 1b). The indenter was adjusted as required using a micromanipulator (Narishige, Japan) to maintain the desired level of applied force (FIG. 1b). Throughout the experiments, the pressure applied to each group was 164±6.7 kPa for the Control Group, 167±26.6 kPa for Experimental Group 1, and 165.2±25.1 kPa for Experimental Group 2. Following the period of pressure application, the leg was unloaded, the nerve-cuffs from both limbs were removed and the skin was sutured. Post-operatively, buprenorphine (0.05 mg/kg) was administered subcutaneously, to alleviate any discomfort.

To test the effect IES alone may have on the stimulated muscles, experiments were conducted in a fourth group of six rats (285±6 g), designated Experimental Group 3 (see FIG. 1e). The experimental procedures previously described were maintained with the exception of no pressure application. The stimulation paradigm utilized was that of Experimental Group 2, with IES being applied to one hind limb of the animal every 5 minutes for a period of 2 hours.

FIG. 1e illustrates the force generated by contraction of muscle in rates in response to intermittent electrical stimulation treatment during 2 hours of treatment. The mean and standard deviation of the force generated by the contraction of the muscle in response to the electrical stimulus during each bout of electrical stimulation over a two hour period are shown for 6 rats (top plot) and 5 rats (bottom plot). Bouts of 10 seconds of stimulation (stimulation ON period) delivered every 5 minutes resulted in force reduction of 25% after 2 hours (top plot). This reduction was not significant and did not significantly affect the effectiveness of the treatment. Bouts of 10 seconds of stimulation (stimulation ON period) delivered every 10 minutes resulted in no force reduction after 2 hours (bottom plot).

### B. Assessment of Deep Tissue Health Using MRI

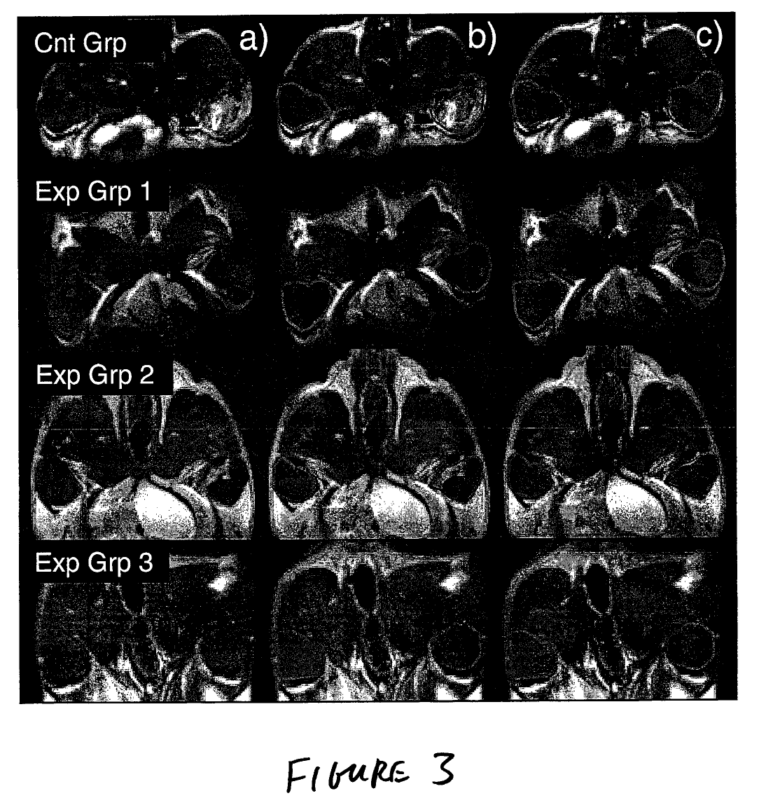

Magnetic resonance imaging was used to obtain an in-vivo assessment of DTI following pressure application and to quantify the effectiveness of IES in preventing such injury (see references 6, 52). Twenty-four hours after the removal of pressure each rat was anesthetized with an intraperitoneal injection of sodium pentobarbital (40 mg/kg). The rat's hind limbs were secured inside a 7-cm diameter birdcage coil and placed inside a 3.0 Tesla magnet (Magnex Scientific PCL). A T₂-weighted spin-echo sequence (echo time (TE)=80 ms, relaxation time (TR)=2000 ms) was employed to detect the presence of edema (as indicated by increased water content) within the quadriceps muscles in both hind limbs of each rat. Data were collected during a 30-minute scanning session and twenty MRI slices (images) were acquired from each rat, with slice thickness of 2 mm and slice separation of 1 mm (every other slice shown in FIG. 2). The acquisition matrix size was 256 pixel×256 pixel within a field of view (FOV) of 120 mm×120 mm, resulting in an in-plane resolution of 0.47 mm×0.47 mm. Both hind legs were imaged in the same slice. MRI slices were obtained in the sagittal, coronal and transverse planes in relation to the rat's femur.

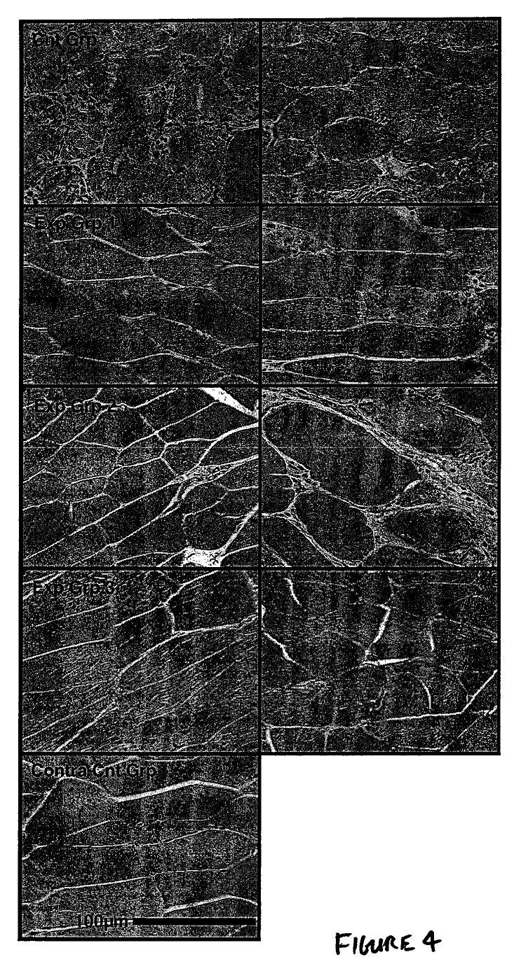

All MRI data were imported to MATLAB 7.0.1 (Mathworks, Natick, Mass., U.S.A.) for analysis using custom-written routines. The left and right quadriceps muscles were manually selected from every slice and all analyses were restricted to the pixels inside these two regions (FIG. 3a). To quantify the amount of increased water content present within the experimental leg from each slice, the signal intensity of each pixel in that leg was compared to a threshold intensity level obtained from the contralateral leg (FIG. 3b). The mean+2*standard deviation in the signal intensity from the quadriceps muscle of the contralateral leg was chosen as the threshold intensity level. If the signal intensity of a pixel in the experimental leg was higher than the threshold, the pixel was considered to have increased water content, or edema (FIG. 3c). A percentage of the affected area relative to the total area of the muscle was obtained from each slice and the total affected volume was calculated for each rat by summing the results from all slices. The threshold was also applied to each control (contralateral) limb from each rat to quantify the amount of increased water content that could be attributed to factors other than the application of pressure or IES, such as the electrode cuff implantation or normal variation in the signal intensity. Results from the untreated contralateral limbs of all 24 rats were designated as the Contralateral Control Group. For measured comparisons between groups both one-way ANOVA and Tukey post-hoc tests were used. All P values less than 0.05 were considered statistically significant.

### C. Histological Assessment

To corroborate the extent of injury in the muscle from the MRI assessment, histological evaluation of the tissue was also performed. Under deep anesthesia (sodium pentobarbital, 40 mg/kg), the animal was transcardially perfused with a formaldehyde (1%)/gluteraldehyde (2.25%) fixative and the quadriceps muscles from both hind limbs were removed. The muscles were photographed, weighed and their volume calculated. The muscle tissue was stored in the same fixative, and subsequently dehydrated through washing in a graded series of ethanol dilutions and embedded in paraffin.

Muscle sections obtained from the region identified by the MR images as containing edema were longitudinally bisected. A 2-3 mm thick longitudinal section was obtained, as well as five 2-3 mm thick transverse sections. A 5 μm slice was obtained from each section and stained with hematoxylin and eosin (H&E).

A veterinary pathologist blinded to the experimental groups performed all histological analyses. A 4.9 mm² area from each slice was assessed to identify muscle fiber necrosis, inflammatory cell infiltration, hemorrhage and tissue mineralization. A necrosis score (0-4) was assigned to each longitudinal slice based on the approximate area exhibiting necrosis out of the slice total area. Subsequently, the transverse slices from each animal were used to confirm the extension of necrosis throughout the muscle. The estimated volume of the muscle affected by necrosis from the histological assessment was compared against the estimated volume of the corresponding muscle affected by edema as calculated from MRI slices. Scoring of histological muscle sections between groups was assessed by a Kruskal-Wallis non-parametric test. All P values less than 0.05 were considered statistically significant. All results are expressed as mean±standard deviation.

### Mechanisms of Action of IES in Human Subjects

### A. Muscle Oxygenation Measurements in Human Subjects

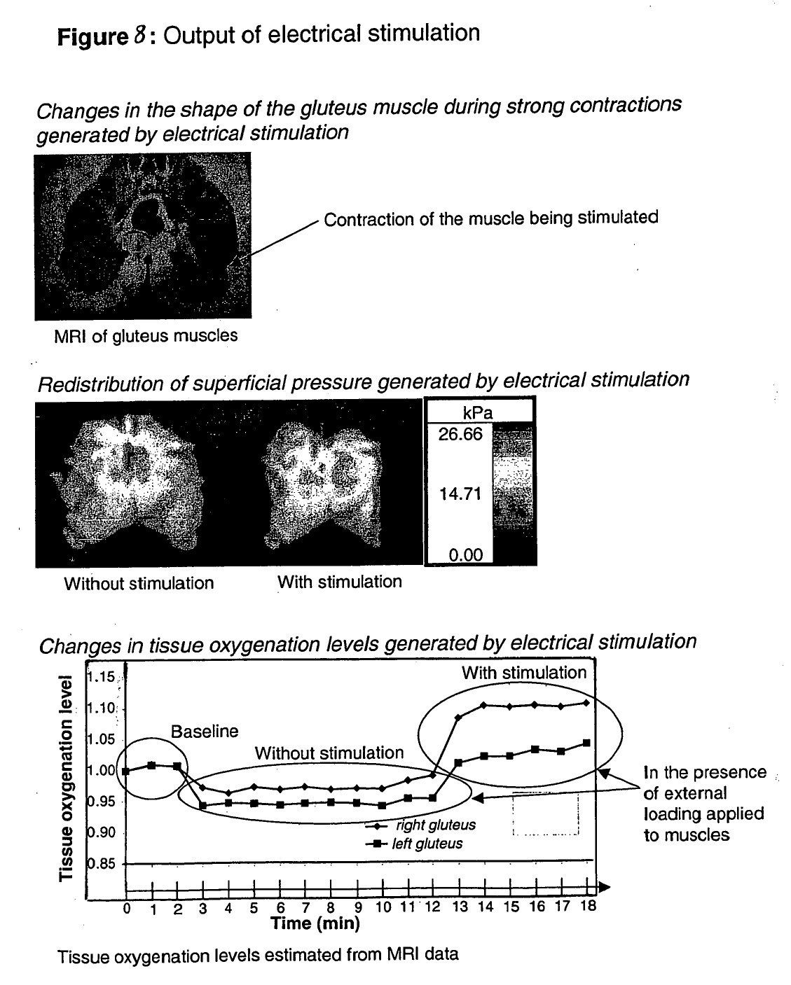

In addition to testing the effectiveness of IES in preventing DTI, we sought to understand the mechanisms of action of IES. An initial experiment was conducted in an able-bodied volunteer (male, 22 yr) to assess changes in tissue oxygenation associated with contractions elicited by IES in an unloaded muscle. The experimental setup is illustrated in FIG. 7. Electrodes were placed on the body and electrical stimulation was delivered to induce muscle contractions. The change in the shape of the muscle during contraction, redistribution of surface pressure and changes in tissue oxygenation were measured using magnetic resonance imaging (MRI) techniques and surface pressure mats. Surface, non-magnetic electrodes were placed over the motor point of the medial gastrocnemius (MG) muscle of one leg. Tissue oxygenation levels were estimated by quantifying changes in the T₂* signal in MR scans of the muscle in which an increase in the T₂* signal is attributed to an influx of oxygenated hemoglobin to the tissue (see references 26, 39). MR scans were acquired with a 1.5 Tesla whole body Siemens Sonata scanner (Siemens Medical Solution, Malvern, Pa.) and a 27-cm diameter transmit/receive knee coil circumscribing the lower leg. A custom-prepared multi-gradient-echo sequence (TR=51.8 ms, 8 TEs ranging from 3.6 ms to 47 ms, single slice, 6 mm slice thickness, flip angle=200, FOV=208 mm×205 mm, readout matrix=160 pixel×158 pixel, in-plane resolution=1.3 mm×1.3 mm) was utilized for all data acquisitions. Baseline levels of oxygenation in MG were obtained as well as simultaneous measurements from the lateral gastrocnemius (LG), medial soleus (MS), and lateral soleus (LS) muscles for comparison. Following the acquisition of baseline scans, successive scans were acquired immediately after 30-s bouts of electrical stimulation delivered through the surface electrodes (biphasic, charge-balanced, constant current, 70 mA, 250 μs, 50 pulses/s).

To mimic a simulated sitting position in which muscles are compressed, albeit around the ischial tuberosities, a second experiment was performed on the gluteus maximus muscles to assess changes in oxygenation levels induced by IES. Surface, non-magnetic electrodes were placed over the motor points of the left and right gluteus maximus muscles of an able-bodied volunteer (male, 26 yr). Due to space limitations within the MRI scanner, which prohibits volunteers from sitting upright, muscle compression during sitting was simulated by adding weight over the pelvis of the person lying supine inside a 1.5 Tesla whole-body scanner. Oxygenation measurements were obtained at: 1) rest, 2) with a 20 kg (30% of body weight) load applied over the pelvis, and 3) with a 20 kg load and IES applied simultaneously.

Surface coils placed below the subject and a multi-gradient-echo sequence (TR=90.3 ms, 20 TEs ranging from 3.8 to 89.6 ms, single slice, 8 mm slice thickness, flip angle=30, FOV=223 mm×397 mm, readout matrix=72 pixel×0.128 pixel, in-plane resolution=3.1 mm×3.1 mm) were utilized for imaging the gluteus in the transverse plane. Three successive 31-s scans were acquired at rest to obtain baseline levels of oxygenation in the left and right gluteus maximus muscles. A 20 kg load was placed over the pelvic region to compress the gluteus muscles and 10 31-s scans were acquired over a 10-minute period of loading. Subsequently, 6 31-s scans were obtained each immediately following a 10-s stimulus bout (biphasic, charge-balanced, constant current, 70 mA, 250 μs, 50 pulses/s, 3-s ramp-up, 3-s ramp-down) applied every minute to the gluteus muscles with the load in place. The stimulation parameters utilized did not cause pain or discomfort to the volunteer.

Magnetic resonance data were imported into MATLAB 7.0.1 (Mathworks, Natick, Mass., U.S.A.) to measure changes in the T₂* signal in each muscle using a mono-exponential non-negative least squares fit routine (see reference 59). A region of interest (ROI) was selected around each target muscle (MG, LG, SM, and SL, or right gluteus maximus, and left gluteus maximus) in each MR slice, and the T₂* levels in each ROI were determined. The T₂* values were normalized to their corresponding baseline levels obtained at rest.

### B. Surface Pressure Measurements

In addition to injury due to ischemic changes, high stress levels and cell deformation have also been associated with tissue damage (see references 7, 8, 11, 35, 36). Ideally, stress levels should be measured at the bone-muscle interface, the place of origin for DTI. However, due to the lack of non-invasive measuring techniques at this deep level, an alternative and commonly used technique is to measure superficial pressure levels at the support surface-skin interface (see reference 5). In order to obtain insight into the effects of IES in reshaping the gluteus maximus muscles, and modifying the surface pressure profiles with each contraction, a single experiment was performed. The experiment was conducted in the same able-bodied volunteer (male, 26 yr), using the same testing conditions as those utilized to assess oxygenation levels in the gluteus maximus muscles: 1) rest, 2) weight, and 3) weight+IES. To elicit contractions in the left and right gluteus maximus muscles, surface electrodes were placed over the motor point of each muscle. The volunteer was placed in a supine position with the buttocks over an X-3 System pressure sensitive mattress (XSensor, Calgary, AB, Canada). Measurements of surface pressure in the sacral region of the buttocks were obtained over a 1-minute period of rest. A 20-kg load, equivalent to 30% of the body weight of the volunteer, was applied over the pelvis to compress the tissue of the buttocks. Surface pressure measurements were acquired for 1 minute under this condition. Electrical stimulation was then applied simultaneously to both gluteus maximus muscles. A series of 3 15-s stimulus bouts (biphasic, charge-balanced, constant current, 70 mA, 250 μs, 50 pulses/s) were applied with the load in place. Changes in surface pressure associated with IES were measured during each bout of stimulation.

### Results

### Effectiveness of IES in Preventing the Formation of DTI

The main objective of this investigation was to determine whether IES is an effective technique for preventing DTI. Our results show that edema and tissue injury can develop after a 2-hour application of constant pressure. In all test groups and at the completion of the study, the skin under the pressure indenter did not exhibit any indication of inflammation or injury, underscoring the difficulty of identifying DTI by visual inspection of the skin.

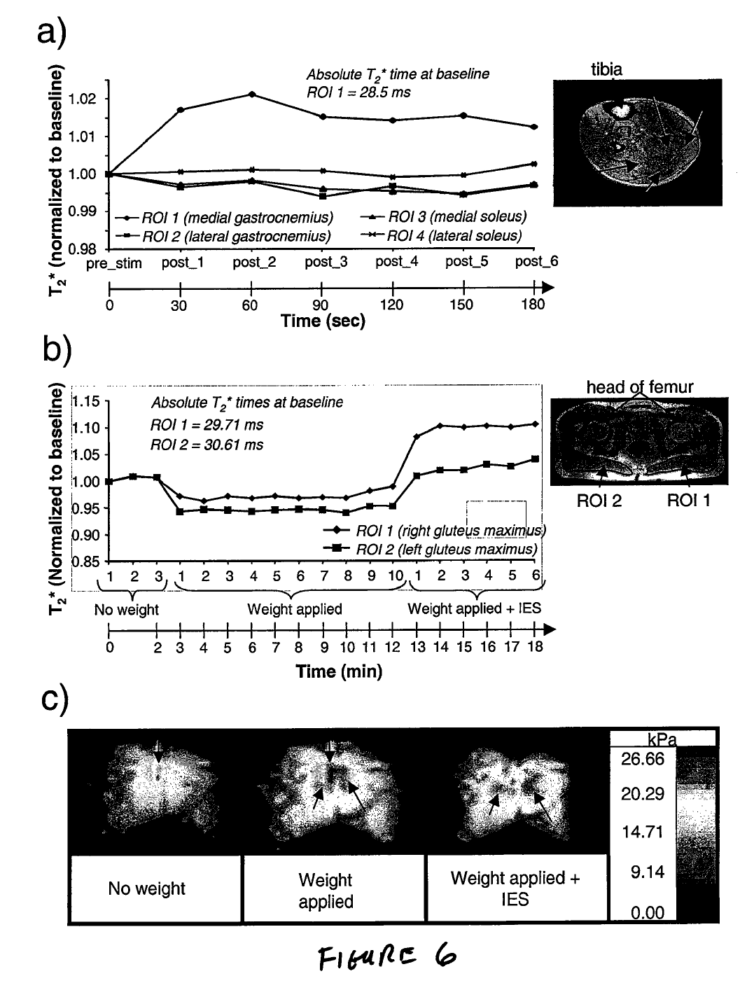

In the Control Group (pressure, No IES), the application of external pressure for 2 hours generated edema in 60±15% of the muscle. In contrast (FIG. 5, left axis, filled circles), Experimental Groups 1 (pressure+IES every 10 min) and 2 (pressure+IES every 5 min) exhibited a significantly reduced region of edema in the muscle, (16±16% for Experimental Group 1 and 25±13% for Experimental Group 2). Experimental Group 3 (No pressure, IES every 5 min) and Contralateral Control Group (untreated contralateral limbs) exhibited a 5±4% and a 5±4% respectively. The extent of increased water content in all three experimental groups was significantly different from that in the Control Group (one-way ANOVA test, p=0.0001), but was not significantly different from each other (Tukey post-hoc test, Exp 1 vs Exp 2, p=0.59; Exp 1 vs Exp 3, p=0.45; Exp 2 vs Exp 3, p=0.06).

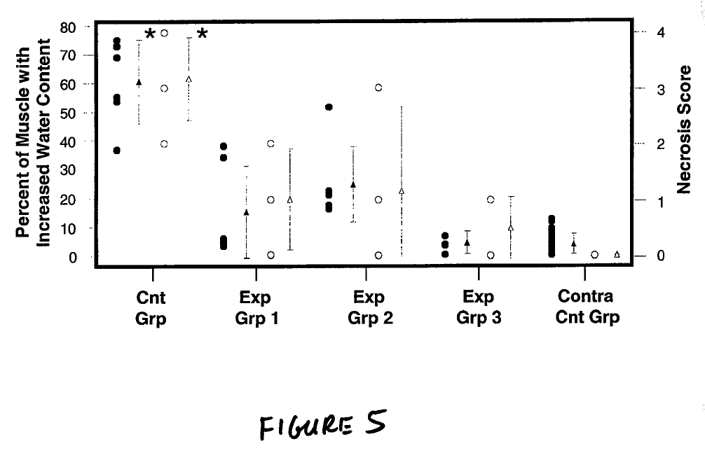

Histological assessment of the quadriceps muscle tissue (FIG. 4; note: the amount of edema observed with MRI correlated well with the amount of necrotic fibers assessed from the histological slides, and all histological sections were viewed at 100× magnification) showed that the severity of muscle injury varied between the control and experimental groups. In general, the lesions within the muscle were characterized by swelling, loss of striations, and fragmentation of muscle fibers. The connective tissue surrounding affected muscle fibers was often infiltrated by numerous neutrophils admixed with smaller numbers of macrophages. Hemorrhage into muscle bundles was most apparent in severely affected tissue. FIG. 5 (right axis, open circles) summarizes the extent of tissue necrosis in the control and experimental groups. The Control Group had the largest extension of necrotic fibers in the tissue with a score of 3.2±0.8. This score represented a necrotic area occupying 25 to 50% of the area analyzed. The extent of tissue necrosis was significantly larger in the Control Group than that in Experimental Group 1, which had a score of 1.0±0.9 (Kruskal-Wallis non-parametric test, p=0.01), representing a necrotic area of less than 10%. Experimental Group 2 also exhibited a significantly smaller area of muscle necrosis than the Control Group (Kruskal-Wallis non-parametric test, p=0.03), with a score of 1.2±1.5, equivalent to a necrotic area between 10% and 20%. The necrosis score was also significantly smaller in Experimental Group 3 (Kruskal-Wallis non-parametric test, p=0.004), with a score of 0.5±0.6. There was no significant difference between all three experimental groups in the amount of necrosis assessed. The infiltration of neutrophils and macrophages, as well as the presence of red blood cells and mineralization of the tissue, were not significantly different between the control and experimental groups.

### Increases in Tissue Oxygenation Due to IES-Elicited Contractions

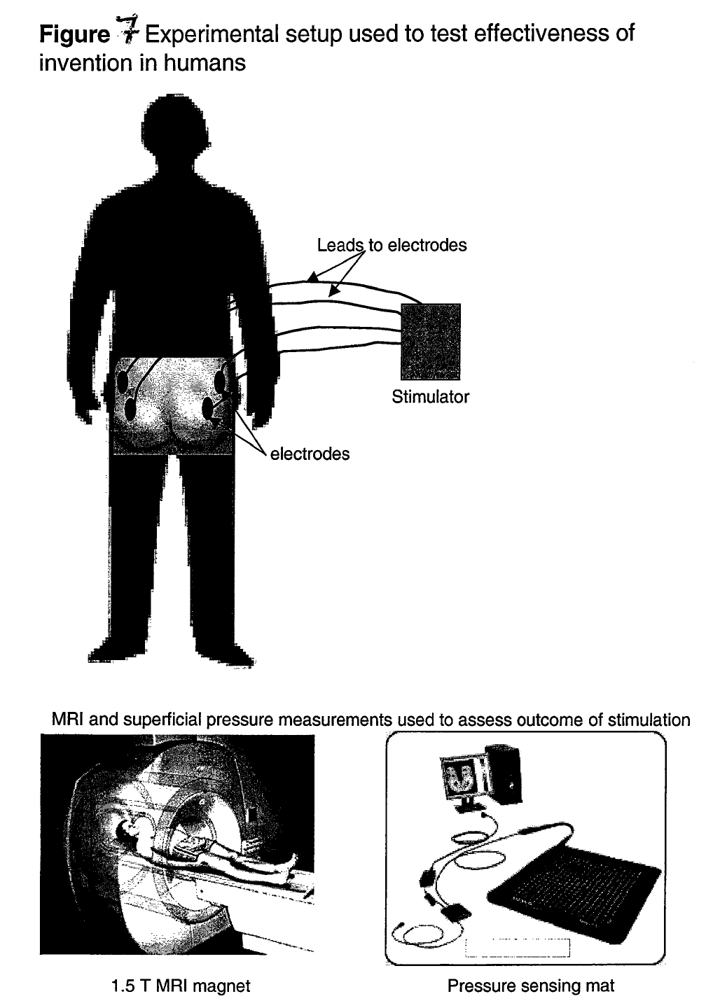

Two experiments were performed with the goal of measuring the changes in tissue oxygenation levels associated with the use of IES. The effects of IES-elicited contractions on muscle oxygenation were first tested in a condition where the muscle was at rest and unloaded. FIG. 6a summarizes the effect of IES on the level of oxygenation in the muscles of the lower leg. Normalized T₂* levels in MG, LG, LS, and MS are shown. Interestingly, IES selectively increased the T₂* level of MG, the stimulated muscle. This increase in oxygenation was maintained throughout the experiment. Oxygenation levels in LG, LS, and MS did not show any change when compared to baseline measurements.

The second experiment measured the increase in tissue oxygenation following IES-elicited contractions of loaded muscles. These loaded muscles had a corresponding reduction in oxygen supply, a situation that represents the state of tissue around the ischial tuberosities in a seated individual. FIG. 6b summarizes the effect of IES on the level of tissue oxygenation in the gluteus maximus muscles in the presence of an external pressure. Normalized T₂* levels in the right and left gluteus maximus muscles are shown for each condition tested (rest, weight, weight+IES). The oxygenation levels in both muscles decreased immediately by ˜4% after the load application; oxygenation remained at this lower level throughout the 10 minutes in which this condition was maintained. Following IES, the oxygenation levels in the muscles increased above the initial baseline levels by ˜6%.

### Changes in Surface Pressure Profiles Due to IES-Elicited Contractions

In a third experiment (FIG. 6c) surface pressure measurements of the buttocks were obtained under the same three conditions previously tested (rest, weight, weight+IES). The average pressure throughout the buttocks at rest was 10.9 kPa, distributed over a 487 mm area. As expected, the region of highest pressure was that surrounding the bony prominence (the sacrum in this case), and exhibited an average pressure of 21.7 kPa.

Following the loading of the pelvis, the average pressure throughout the buttocks increased to 13.9 kPa and was distributed over a 511 mm² area. The average pressure in the region around the sacrum increased to 25.8 kPa. Simultaneous bilateral application of IES to the loaded (compressed) gluteus maximus muscles induced contractions which reconfigured the shape of the muscles. The average pressure throughout the buttocks became 14.3 kPa distributed over an area of 424 mm². However, the average pressure around the sacrum was reduced to 19.5 kPa, a level lower than that seen even during the rest condition.

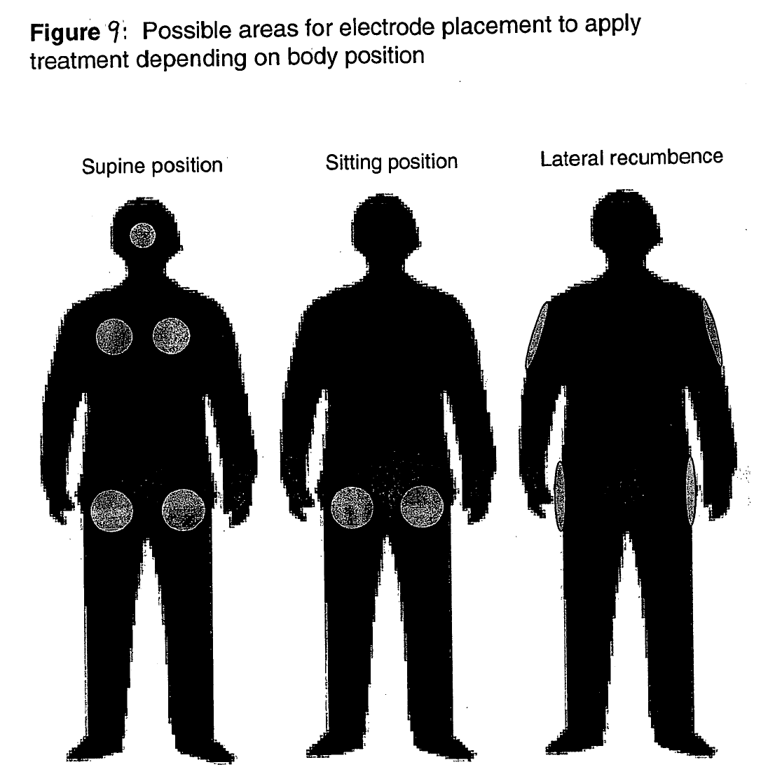

FIG. 8 illustrates an MRI of the left and right gluteus muscles demonstrating the changes in muscle shape during contractions induced by electrical stimulation (top). It also shows the redistribution of surface pressure (middle) and the increase in tissue oxygenation (bottom) during electrical stimulation.

### Discussion

### Effectiveness of IES in Preventing the Formation of DTI

Several studies have reported the beneficial effects of both alternating and direct current electrical stimulation for healing chronic wounds, including pressure ulcers (see references 4, 17, 21, 25, 43, 51, 58). The consensus is that when combined with traditional treatments, electrical stimulation improves wound healing. Very few studies however, have investigated electrical stimulation alone as a method for preventing the formation of pressure ulcers.

Levine et al. first proposed using electrical stimulation to prevent pressure ulcers and measured the effect of electrical muscle stimulation on 1) pressure at the seating interface (see reference 32), 2) muscle shape (see reference 33), and 3) blood flow (see reference 34). Their results indicated that during each contraction of the gluteus muscles 1) the superficial pressure surrounding the ischial tuberosities was reduced; 2) the shape of the compressed muscle was modified; and 3) blood flow increased in the stimulated muscle. Based on these observations, it was suggested that electrical stimulation might be an effective technique to prevent pressure ulcers.

Following the seminal study of Levine et al., Rischbieth et al (see reference 44) and Bogie et al (see reference 4) reported that an increase in muscle mass was achieved through long-term electrical stimulation. The increase in muscle mass was suggested to provide individuals with improved cushioning, which in turn, could prolong the time they can remain seated. Recently, Bogie et al (see reference 5) analyzed the long-term effects of electrical stimulation of the gluteus muscles in one individual with spinal cord injury. Measurements of surface interface pressure, transcutaneous oxygen levels, and muscle thickness were similar to observations previously reported by Levine (see references 32-34), Rischbieth (see reference 44), and Bogie (see reference 4). It was also determined that any benefits gained during the period of electrical stimulation were abolished once the electrical stimulation was discontinued. While the evidence from these studies suggested the potential effectiveness of IES in preventing the formation of pressure ulcers, heretofore no study had investigated the effects of IES on the integrity of deep muscle exposed to constant pressure.

The present study examined the efficacy of IES in preventing DTI in a rat model and its mechanism of action in human volunteers. Our results show, that within defined parameters of electrical stimulation, a considerable reduction in DTI was observed. Traditionally, tissue injury generated by ischemia following long periods of tissue compression, has been considered the principal etiological factor behind pressure ulcers (see references 27-29). Within this precept, more frequent stimulation should restore tissue oxygenation in the tissue to normal or near-normal levels, potentially eliminating tissue injury caused by ischemia. The finding that there was no significant difference between our experimental groups (IES every 10 minutes vs. 5 minutes) could indicate that the beneficial effects of an increase in oxygenation to the tissue may have reached their threshold when stimulation occurred every 10 minutes. It is possible that the amount of damage observed in both experimental groups could be attributed to damage generated directly by the high stress levels at the bone-muscle interface and excessive cell deformation, a factor that was further exaggerated in our experimental set up due to the fixation of the hind limb which led to an increase, rather than a decrease, in focal pressure during the IES-induced contractions (evident in the increases in recorded force in FIG. 1b). Although the application of pressure to the rats' limbs was done outside the MRI scanner, utmost care was taken in the placement of the indenter, such that it was as centered as possible over the QM and the femur.

Comparison of Experimental Group 3 and the Contralateral Control Group demonstrated that the use of IES as frequently as every 5 minutes does not cause an increase in the water content of the muscle. The minimal amount of water content identified in the Contralateral Control Group, as calculated in this study, indicates that ˜5% of the tissue water content quantified in the Control Group and Experimental Groups 1 and 2 was not caused by the load application.

It has been suggested that high stress levels at the bone-muscle interface is a primary factor in the development of pressure ulcers (see references 7, 8, 11, 35, 36), but the extent of tissue injury that is associated with these mechanical forces (shear and stress) has yet to be determined. Although complete elimination of DTI has not been achieved, our results suggest that IES delivered every 10 minutes is sufficient to reduce greatly the extent of damage in deep tissue exposed to constant external pressure.

None of the rats in this study showing indications of DTI displayed injury to the overlying skin. This emphasizes that skin appearance is a poor indicator of deep tissue health, and supports the need for other alternative methods to detect DTI. The results of this study, as well as those reported previously by Bosboom et al (see reference 6) and Stekelenburg et al (see reference 52), show that MRI is an effective tool for the detection of muscle edema associated with the presence of DTI, even when injury occurs in muscles as small as those in the rat hind limbs (FIG. 3a). Although MRI currently may not be ideal for screening patients with DTI due to cost and availability, in situations where an individual is considered to be at high risk of developing an ulcer or has a long history of ulcer development, it might be necessary to perform periodic screenings. Identifying DTI before it fully evolves into a pressure ulcer would not only have a significant beneficial impact on the health and quality of life of the individual, but could greatly reduce costs associated with further medical and surgical treatments.

### Mechanisms of Action of IES

Our results demonstrated that the levels of available oxygen in the tissue of gluteus maximus were reduced immediately after compressing the muscles (FIG. 6). However, instantly following the first IES-induced contraction of the muscles, the levels of tissue oxygen increased. This increase was greater than baseline levels, and was most likely caused by reactive hyperemia, a process in which there is an increase in blood flow into the capillaries after brief periods of occlusion (see reference 38). This increase in oxygenation was maintained after each of the 6 IES-induced contractions. While oxygenation levels in the unloaded medial gastrocnemius muscle also increased with IES, the increase was less than that in the gluteal measurements. This may be due to the fact that blood flow to the medial gastrocnemius muscle was not altered, and consequently oxygenation levels were already at normal levels.

While periodical increases in tissue oxygenation should have the beneficial effect of negating tissue injury associated with ischemia-reperfusion, pressure relief is still needed to prevent further damage from persistent high stress levels of muscle cells. Our results demonstrated that IES of the compressed gluteus muscles reconfigured the shape of the muscles and distributed the pressure laterally in the buttocks. The net result was a periodical relief of the superficial pressure around the bony prominence and reduction in the overall pressure throughout the buttocks. The use of superficial pressure measurements combined with recently developed finite element models (see references 37, 40) of the gluteal muscles which can estimate the stress levels at the bone-muscle interface, could provide a more accurate tool for predicting the risk of developing DTI.

### Example No. 2

Experiments were conducted in seated volunteers to evaluate the effect of various parameters of IES on: 1) the redistribution of surface pressure during contraction, 2) changes in the shape of the gluteus maximums muscles around the ischial tuberosities, and 3) changes in tissue oxygenation. Surface pressure mats and magnetic resonance imaging (MRI) techniques were used for the measurements.

Five (5) able-bodied volunteers with intact spinal cord and four (4) volunteers with spinal cord injury (SCI) participated in the study. Four (4) IES patterns were tested between the two groups of volunteers as described in Table 1 below. Electrical stimulation was provided through surface electrodes placed on the motor points of the gluteus maximus muscles of both legs in all volunteers.

TABLE 1
IES parameters tested in volunteers with intact and SCI study participants

| | IES ON:OFF | Stimulation characteristics | |
|---|---|---|---|
| Volunteer | periods | during ON period | Stimulation parameters |
| Intact | 10 (sec):7 (min) | continuous | 200 μs pulses, 20-120 mA, |
| | | | 40-50 pulses/s |
| | 10 (sec):7 (min) | pulsatile (3 sec on:2 sec off:3 | 200 μs pulses, 20-120 mA, |
| | | sec on:2 sec off:3 sec on) | 40-50 pulses/s |
| SCI | 7 (sec):10 (min) | continuous | 200 μs pulses, 20-120 mA, |
| | | | 40-50 pulses/s |
| | 13 (sec):10 (min) | continuous | 200 μs pulses, 20-120 mA, |
| | | | 40-50 pulses/s |

Redistributions in surface pressure with IES were assessed with the volunteers seated in a regular office chair (intact) or a wheelchair containing a standard pressure relief cushion (SCI). A pressure mat containing a 36×36 array of sensors was placed between the volunteers and the sitting surface. A map of the surface pressure was obtained during the OFF period of IES and compared to that obtained during the ON period.

Surface pressure was highest around the ischial tuberosities (the bones we sit on) in both intact and SCI individuals during the OFF period of IES. During the ON period, contractions of the gluteus maximus muscles generated a redistribution in surface pressure in both intact and SCI volunteers. There were decreases in surface pressure around the high-risk ischial tuberosity regions that are most susceptible to the formation of pressure ulcers. Concomitant increases in pressure in the surrounding areas, low-risk regions, were seen.

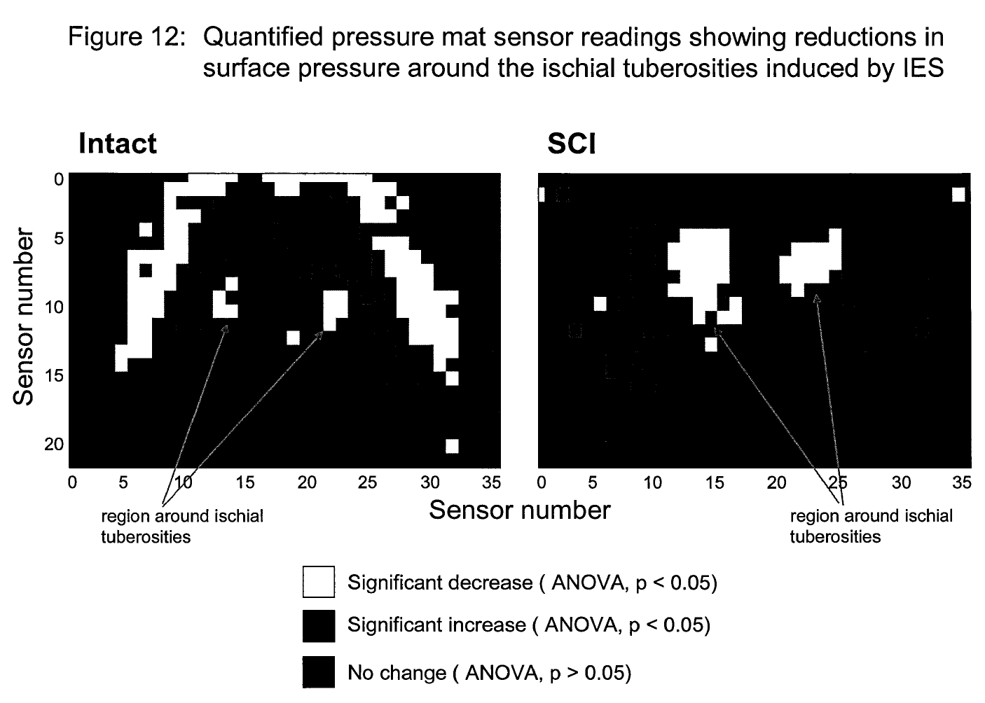

The redistribution in surface pressure produced by IES was quantified by comparing the changes in readings of each of the sensors embedded within the pressure mat during the stimulation ON and OFF periods. FIG. 12 shows a typical example of the distribution of sensors showing statistically significant changes in pressure readings between the ON and OFF periods of IES. Sensors with a significant reduction in pressure readings during the ON period of IES relative to the OFF period are shown in white, those with a significant increase are shown in grey, and those with no significant change are shown in black. During the ON period of IES, significant reductions in pressure were obtained around the ischial tuberosities in both able-bodied and SCI volunteers, regardless of the sitting surface (regular chair vs. wheelchair with pressure relief cushion).

During these experiments, able-bodied volunteers as well as those with SCI who had some preserved sensation around the gluteal region (n=2) reported that IES relieved their discomfort due to long durations of sitting. Furthermore, the relief was sustained for several minutes after the ON period of IES. In comparison to standard clinical practices such as wheelchair push-ups, the volunteers reported that IES provided more relief of discomfort due to sitting and for longer durations. Able-bodied volunteers preferred the continuous mode of stimulation during the ON period of IES over the pulsatile, even though they reported that both patterns produced a similar level of relief of discomfort due to sitting. Because of their altered sensation, the SCI volunteers could not subjectively compare the level of relief produced by the two durations of the continuous mode of stimulation during the ON period of IES (7 vs. 13 seconds).

To investigate the changes in the shape of the muscle produced by IES as well as changes in oxygenation levels of deep tissue, the volunteers were transferred to a custom built MRI-compatible apparatus. This apparatus positioned the volunteers in a manner that mimicked a sitting posture and produced similar surface pressure profiles to those obtained while sitting in a chair/wheelchair.

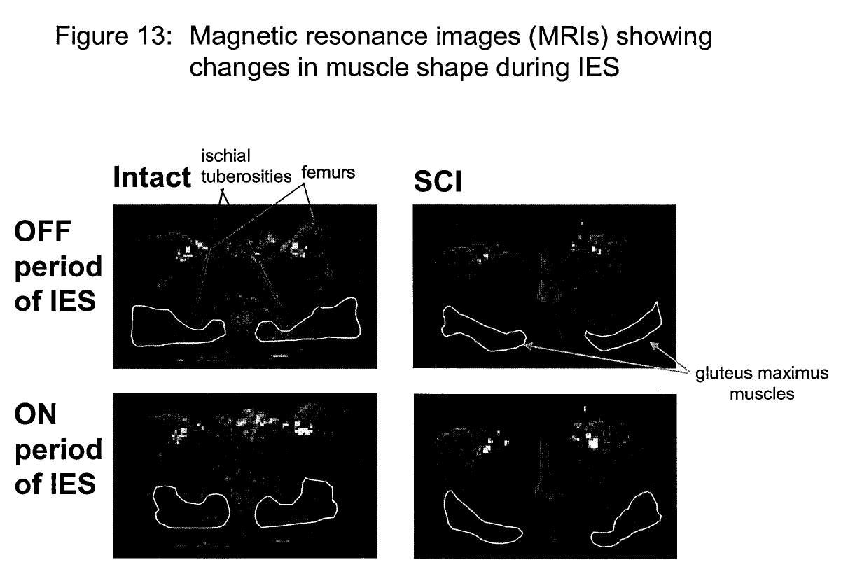

FIG. 13 provides T₂-weighted MRI scans which show the shape of the gluteus maximus muscles at rest (OFF period of IES) and during contraction (ON period of IES) for able-bodied (intact, left) and injured (SCI, right) volunteers. Substantial changes in muscle shape were seen in the intact volunteers (FIG. 13, left). Changes in muscle shape were also seen even in the much atrophied muscles of SCI volunteers (FIG. 13, right). These changes explain the redistributions in surface pressure seen in FIG. 12, and demonstrate that redistributions in internal pressure are also obtained by IES.

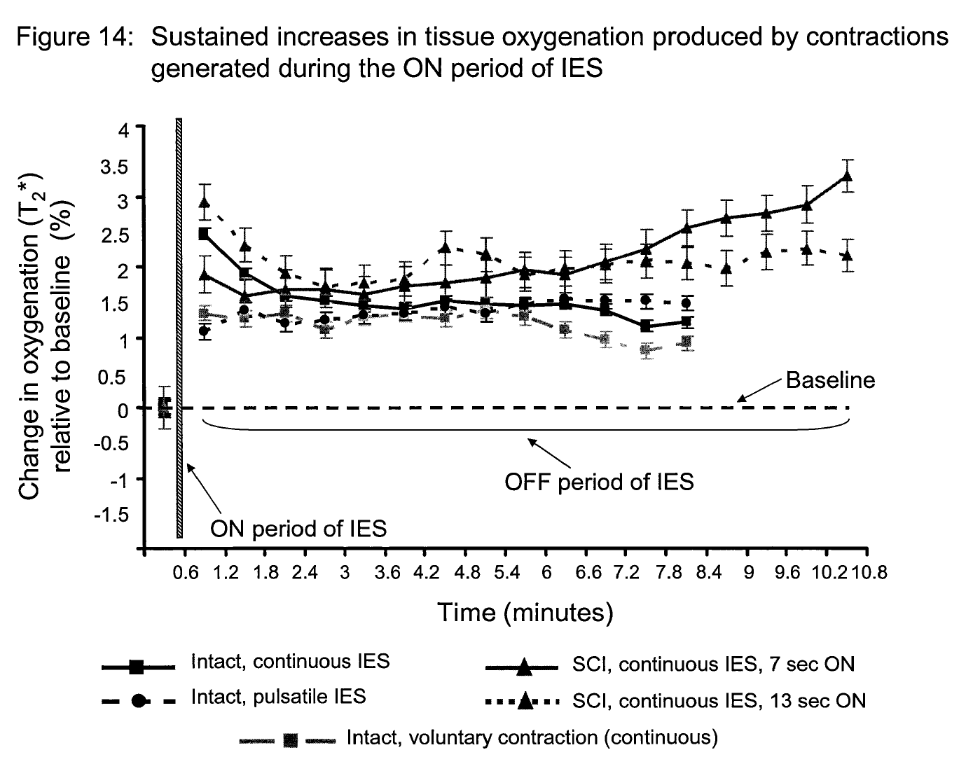

To assess the changes in tissue oxygenation, T₂* MRI images were obtained and alterations in the signal intensity in the gluteus maximus muscles due to IES were quantified as previously described (pages 31-32, FIG. 6 a, b). FIG. 14 summarizes the changes in tissue oxygenation (mean±standard deviation) seen in intact and SCI volunteers, and in response to the four (4) patterns of IES tested (Table 1). To compare the changes in oxygenation in response to contractions produced by IES and voluntary activation, the able-bodied volunteers were also asked to contract their gluteus maximus muscles voluntarily and to hold the contraction for 10 seconds (mimicking the sec ON, continuous, IES pattern).

In all cases, significant increases (ANOVA, p<0.05) in tissue oxygenation were seen following the ON period of IES. These increases were at times more prominent than those produced by voluntary contraction. Furthermore, the increases in oxygenation were sustained for up to 10 minutes (longest IES OFF period tested to date), which explains the sustained relief from discomfort reported by the volunteers during the surface pressure measurements described above. Very importantly, the pattern of tissue oxygenation observed in volunteers with SCI was similar to that seen in intact volunteers, despite their substantially atrophied muscles. While direct measurements of blood flow or oxygen were not obtained, the increases in oxygenation (1-3% increase in T₂* signal intensity) are estimated to reflect a 15-45% increase in blood flow (see reference 62) in the gluteus maximus muscles.

Some differences were observed in the level of tissue oxygenation produced by the various patterns of IES. First, the continuous pattern of stimulation during the ON period of IES produced larger increases in oxygenation immediately following the cessation of stimulation compared to the pulsatile pattern. However, by ˜3 minutes within the OFF period of IES, the oxygenation levels were similar for both the continuous and pulsatile patterns. Second, longer stimulation durations during the ON period of IES produce larger increases in oxygenation immediately following the cessation of stimulation. However, by 2 minutes within the OFF period of IES, the oxygenation levels were similar for all durations of the ON period of IES tested (i.e., 7, and 13 seconds). Third, the changes in tissue oxygenation produced by the pulsatile pattern of stimulation during the ON period of IES were similar in profile to those produced by voluntary contraction.

In conclusion, the experiments in the seated individuals (intact and SCI) demonstrated that IES is an effective means for redistributing surface pressure, changing muscle shape, and producing sustained increases in deep tissue oxygenation. All tested patterns of IES were effective in achieving these outcomes. Therefore, IES may provide a powerful means for prophylactically preventing the formation of pressure ulcers originating at deep bone-muscle interfaces.

Although the disclosure describes and illustrates various embodiments of the invention, it is to be understood that the invention is not limited to these particular embodiments. Many variations and modifications will now occur to those skilled in the art of headwear. For full definition of the scope of the invention, reference is to be made to the appended claims.

### REFERENCES

1. Agency for Health Care Policy & Research PfPUT. Clinical Practice Guideline Number 15. Treatment of Pressure Ulcers. US Department of Health & Human Services, Public Health Service AHCPR Publication No 95-0652, 1994.
2. Ankrom M A, Bennett R G, Sprigle S, Langemo D, Black J M, Berlowitz D R, Lyder C H, and Panel. NPUA. Pressure-related deep tissue injury under intact skin and the current pressure ulcer staging systems. Advances in Skin and Wound Care 18: 35-42, 2005.
3. Black J M and Panel. NPUA. Moving toward consensus on deep tissue injury and pressure ulcer staging. Advances in Skin and Wound Care 18: 415-421, 2005.
4. Bogie K M, Reger S I, Levine S P, and Sahgal V. Electrical stimulation for pressure sore prevention and wound healing. Assistive Technology 12: 50-66, 2000.
5. Bogie K M, Wang X, and Triolo R J. Long-term prevention of pressure ulcers in high-risk patients: a single case study of the use of gluteal neuromuscular electric stimulation. Archives of Physical Medicine & Rehabilitation 87: 585-591, 2006.
6. Bosboom E M H, Bouten C V, Oomens C W, Baaijens F P, and Nicolay K. Quantifying pressure sore-related muscle damage using high resolution MRI. Journal of Applied Physiology 95: 2235-2240, 2003.
7. Bouten C V, Oomens C W, Baaijens F P, and Bader D L. The etiology of pressure ulcers: skin deep or muscle bound? Archives of Physical Medicine and Rehabilitation 84: 616-619, 2003.
8. Breuls R G M, Bouten C V, Oomens C W, Bader D L, and Baaijens F P. A theoretical analysis of damage evolution in skeletal muscle tissue with reference to pressure ulcer development. Journal of Biomedical Engineering 125: 902-909, 2003.
9. Collins F. Sitting: pressure ulcer development. Nursing Standard 15: 54-58, 2001.
10. Conine T A, Choi A K, and Lim R. The user-friendliness of protective support surfaces in prevention of pressure sores. Rehabilitation Nursing 14: 261-263, 1989.
11. Daniel R K, Priest D L, and Wheatley D C. Etiologic factors in pressure sores: an experimental model. Archives of Physical Medicine & Rehabilitation 62: 492-498, 1981.
12. Edlich R F, Winters K L, Woodard C R, Buschbacher R M, Long W B, Gebhart J H, and Ma E K Pressure ulcer prevention. Journal of Long-term Effects of Medical Implants 14: 285-304, 2004.
13. Fennegan D. Positive living or negative existence? Nursing Times 15: 51-54, 1983.
14. Finestone H M, Levine S P, Carlson G A, Chizinsky K A, and Kett R L. Erythema and skin temperature following continuous sitting in spinal cord injured individuals. Journal of Rehabilitation Research & Development 28: 27-32, 1991.
15. Garber S L and Dyerly L R. Wheelchair cushions for persons with spinal cord injury: an update. American Journal of Occupational Therapy 45: 550-554, 1991.
16. Garber S L and Krouskop T A. Body build and its relationship to pressure distribution in the seated wheelchair patient. Archives of Physical Medicine & Rehabilitation 63: 17-20, 1982.
17. Gardner S E, Frantz R A, and Schmidt F L. Effect of electrical stimulation on chronic wound healing: a meta-analysis. Wound Repair and Regeneration 7: 495-503, 1999.
18. Gefen A, Gefen N, Linder-Ganz E, and Margulies S S. In vivo muscle stiffening under bone compression promotes deep pressure sores. Journal of Biomechanical Engineering 127: 512-524, 2005.
19. Gilsdorf P, Patterson R, and Fisher S. Thirty-minute continuous sitting force measurements with different support surfaces in the spinal cord injured and able-bodied. Journal of Rehabilitation Research & Development 28: 33-38, 1991.
20. Grace P A. Ischaemia-reperfusion injury. British Journal of Surgery 81: 637-647, 1994.
21. Griffin J W, Tooms R E, Mendius R A, Clifft J K, Vander Zwaag R, and el-Zeky F. Efficacy of high voltage pulsed current for healing of pressure ulcers in patients with spinal cord injury. Physical Therapy 71: 433-442, 1991.
22. Gunningberg L, Lindholm C, Carlsson M, and Sjoden P. Effect of visco-elastic foam mattresses on the development of pressure ulcers in patients with hip fractures. Journal of Wound Care 9: 455-460, 2000.
23. Gute D C, Ishida T, Yarimizu K, and Korthuis R J. Inflammatory responses to ischemia and reperfusion in skeletal muscle. Molecular & Cellular Biochemistry 179: 169-187, 1998.
24. Guthrie R H and Goulian D. Decubitus ulcers: Prevention and Treatment. Geriatrics: 67-71, 1973.
25. Houghton P E, Kincaid C B, Lovell M, Campbell K E, Keast D H, Woodbury M G, and Harris K A. Effect of electrical stimulation on chronic leg ulcer size and appearance. Physical Therapy 83: 17-28, 2003.
26. Jordan B F, Kimpalou J Z, Beghein N, Dessy C, Feron O, and Gallez B. Contribution of oxygenation to BOLD contrast in exercising muscle. Magnetic Resonance in Medicine 52: 391-396, 2004.
27. Kosiak M. Etiology and pathology of ischemic ulcers. Archives of Physical Medicine & Rehabilitation 40: 62-69, 1959.
28. Kosiak M. Etiology of decubitus ulcers. Archives of Physical Medicine & Rehabilitation 42: 19-29, 1961.
29. Kosiak M, Kubicek W G, Olson M, Danz J N, and Kottke F J. Evaluation of pressure as factor in production of ischial ulcers. Archives of Physical Medicine & Rehabilitation 39: 623-629, 1958.
30. Krause J S and Broderick L. Patterns of recurrent pressure ulcers after spinal cord injury: identification of risk and protective factors 5 or more years after onset. Archives of Physical Medicine & Rehabilitation 85: 1257-1267, 2004.
31. Labbe R, Lindsay T, and Walker P M. The extent and distribution of skeletal muscle necrosis after graded periods of complete ischemia. Journal of Vascular Surgery 6: 152-157, 1987.
32. Levine S P, Kett R L, Cederna P S, Bowers L D, and Brooks S V. Electrical muscle stimulation for pressure variation at the seating interface. Journal of Rehabilitation Research & Development 26: 1-8, 1989.
33. Levine S P, Kett R L, Cederna P S, and Brooks S V. Electric muscle stimulation for pressure sore prevention: tissue shape variation. Archives of Physical Medicine & Rehabilitation 71: 210-215, 1990.
34. Levine S P, Kett R L, Gross M D, Wilson B A, Cederna P S, and Juni J E. Blood flow in the gluteus maximus of seated individuals during electrical muscle stimulation. Archives of Physical Medicine & Rehabilitation 71: 682-686, 1990.
35. Linder-Ganz E, Engelberg S, Scheinowitz M, and Gefen A. Pressure-time cell death threshold for albino rat skeletal muscles as related to pressure sore biomechanics. Journal of Biomechanics 39: 2725-2732, 2006.
36. Linder-Ganz E and Gefen A. Mechanical compression-induced pressure sores in rat hindlimb: muscle stiffness, histology, and computational models. Journal of Applied Physiology 96: 2034-2039, 2004.
37. Linder-Ganz E, Shabshin N, Itzchak Y, and Gefen A. Assessment of mechanical conditions in sub-dermal tissues during sitting: A combined experimental-MRI and finite element approach. Journal of Biomechanics, 2006.
38. Mollison H L, McKay W P, Patel R H, Kriegler S, and Negraeff O E. Reactive hyperemia increases forearm vein area. Canadian Journal of Anaesthesia 53: 759-763, 2006.
39. Noseworthy M D, Bulte D P, and Alfonsi J. BOLD magnetic resonance imaging of skeletal muscle. Seminars in Musculoskeletal Radiology 7: 307-315, 2003.
40. Oomens C W, Bressers O F, Bosboom E M, Bouten C V, and Bader D L. Can loaded interface characteristics influence strain distributions in muscle adjacent to bony prominences? Computer Methods in Biomechanic & Biomedical Engineering 6: 171-180, 2003.
41. Peirce S M, Skalak T C, and Rodeheaver G T. Ischemia-reperfusion injury in chronic pressure ulcer formation: a skin model in the rat. Wound Repair and Regeneration 8: 68-76, 2000.
42. Raghavan P, Raza W A, Ahmed Y S, and Chamberlain M A. Prevalence of pressure sores in a community sample of spinal injury patients. Clinical Rehabilitation 17: 879-884, 2003.
43. Reger S I, Hyodo A, Negami S, Kambic H E, and Sahgal V. Experimental wound healing with electrical stimulation. Artificial Organs 23: 460-462, 1999.
44. Rischbieth H, Jelbart M, and Marshall R. Neuromuscular electrical stimulation keeps a tetraplegic subject in his chair: a case study. Spinal Cord 36: 443-445, 1998.
45. Russell L. Pressure ulcer classification: defining early skin damage. British Journal of Nursing 11: S33-34, S36, S38, S40-41, 2002.
46. Russell L J, Reynolds T M, Park C, Rithalia S, Gonsalkorale M, Birch J, Torgerson D, Iglesias C, and Group. P-S. Randomized clinical trial comparing 2 support surfaces: results of the Prevention of Pressure Ulcers Study. Advances in Skin and Wound Care 16: 317-327, 2003.
47. Sagach V F, Kindybalyuk A M, and Kovalenko T N. Functional hyperemia of skeletal muscle: role of endothelium. Journal of Cardiovascular Pharmacology 20: S170-S175, 1992.
48. Salcido R, Fisher S B, Donofrio J C, Bieschke M, Knapp C, Liang R, LeGrand E K, and Carney J M. An animal model and computer-controlled surface pressure delivery system for the production of pressure ulcers. Journal of Rehabilitation Research & Development 32: 149-161, 1995.
49. Salzberg C A, Byrne D W, Cayten C G, van Niewerburgh P, Murphy J G, and Viehbeck M. A new pressure ulcer risk assessment scale for individuals with spinal cord injury. American Journal of Physical Medicine & Rehabilitation 75: 96-104, 1996.
50. Seymour R J and Lacefield W E. Wheelchair cushion effect on pressure and skin temperature. Archives of Physical Medicine & Rehabilitation 66: 103-108, 1985.
51. Stefanovska A, Vodovnik L, Benko H, and Turk R. Treatment of chronic wounds by means of electric and electromagnetic fields. Part 2. Value of FES parameters for pressure sore treatment. Medical and Biological Engineering and Computing 31: 213-220, 1993.
52. Stekelenburg A, Oomens C W, Strijkers G J, Nicolay K, and Bader D L. Compression-induced deep tissue injury examined with magnetic resonance imaging and histology. Journal of Applied Physiology 100:1946-1954, 2006.
53. Swarts A E, Krouskop T A, and Smith D R. Tissue pressure management in the vocational setting. Archives of Physical Medicine & Rehabilitation 69: 1988, 1988.
54. Thomas D R. Are all pressure ulcers avoidable? Journal of the American Medical Directors Association 4: S43-S48, 2003.
55. Tsuji S, Ichioka S, Sekiya N, and Nakatsuka T. Analysis of ischemia-reperfusion injury in a microcirculatory model of pressure ulcers. Wound Repair and Regeneration 13: 209-215, 2005.
56. Tupling R, Green H, Senisterra G, Lepock J, and McKee N. Effects of ischemia on sarcoplasmic reticulum Ca(2+) uptake and Ca(2+) release in rat skeletal muscle. American Journal of Physiology Endocrinology & Metabolism 281: E224-E232, 2001.
57. Tupling R, Green H, Senisterra G, Lepock J, and McKee N. Ischemia-induced structural change in SR Ca2+-ATPase is associated with reduced enzyme activity in rat muscle. American Journal of Physiology Regulatory Integrative & Comparative Physiology 281: R1681-R1688, 2001.
58. Vodovnik L and Karba R. Treatment of chronic wounds by means of electric and electromagnetic fields. Part 1. Literature review. Medical and Biological Engineering and Computing 30: 257-266, 1992.
59. Whittall K P and Mackay A L. Quantitative interpretation of NMR relaxation data. Journal of Magnetic Resonance 84: 134-152, 1989.
60. Woolsey R M and McGarry J D. The cause, prevention, and treatment of pressure sores. Neurologic Clinics 9: 797-808, 1991.
61. Zanca J M, Brienza D M, Berlowitz D, Bennett R G, Lyder C H, and N.P.U.A.P. Pressure ulcer research funding in America: creation and analysis of an on-line database. Advances in Skin and Wound Care 16: 190-197, 2003.
62. Ledermann H P, Heidecker H G, Schulte A C, Thalhammer C, Aschwanden M, Jaeger K A, Scheffler K, Bilecen D. Calf Muscles Imaged at BOLD MR: Correlation with TcPO2 and Flowmetry Measurements during Ischemia and Reactive Hyperemia—Initial Experience. Radiology 241: 477-484, 2006.

What is claimed is:

1. A method for mitigating or preventing formation of pressure ulcers in a patient by transmitting an electrical stimulus to a skin portion of a patient sufficient to effect contraction of a muscle, wherein the method includes a first mode of operation and a second mode of operation, comprising:
over a period of at least about one hour, continuously switching between the first mode of operation and the second mode of operation;
wherein, for each instance of the first mode of operation, the first mode of operation lasts a respective predetermined stimulus time duration, and a respective operative electrical stimulus, sufficient to effect contraction of a muscle, is transmitted to the skin portion, thereby effecting contraction of the muscle, during the entire, or substantially the entire, respective predetermined stimulus time duration;
and wherein, for each instance of the second mode of operation, the second mode of operation lasts a respective predetermined relaxation time duration, and the muscle is relaxed during the entire, or substantially the entire, respective predetermined relaxation time duration.

2. The method as claimed in claim 1, wherein the respective predetermined relaxation time duration for each instance of the second mode of operation is at least five (5) minutes.

3. A method for mitigating or preventing formation of pressure ulcers in a patient, comprising:
over a period of at least about one hour, transmitting a plurality of intermittent transmissions of an electrical stimulus, each one of the plurality of intermittent transmissions sufficient to effect contraction of a muscle, to a skin portion of the patient, thereby effecting contraction of the muscle;
wherein a respective time interval is provided between each pair of successive intermittent transmissions of the plurality of intermittent transmissions, such that there is a plurality of respective time intervals;
and wherein the muscle is relaxed during the entirety, or the substantial entirety, of each one of the plurality of respective time intervals.

4. The method as claimed in claim 3, wherein each one of the plurality of respective time intervals is at least five (5) minutes.

5. Use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions is transmitted over a period of at least about one hour, and wherein a respective time interval is provided between each pair of successive intermittent transmissions of the plurality of intermittent transmissions, such that there is a plurality of respective time intervals, and wherein the muscle is relaxed during the entirety, or the substantial entirety, of each one of the plurality of respective time intervals.

6. A method for mitigating or preventing formation of pressure ulcers in a patient by transmitting an electrical stimulus to a skin portion of a patient sufficient to effect contraction of a muscle, wherein the method includes a first mode of operation and a second mode of operation, comprising:
continuously switching between the first mode of operation and the second mode of operation;
wherein, for each instance of the first mode of operation, the first mode of operation lasts a respective predetermined stimulus time duration, and a respective operative electrical stimulus, sufficient to effect contraction of a muscle, is transmitted to the skin portion, thereby effecting contraction of the muscle, during the entire, or substantially the entire, respective predetermined stimulus time duration;
and wherein, for each instance of the second mode of operation, the second mode of operation lasts a respective predetermined relaxation time duration, and the muscle is relaxed during the entire, or substantially the entire, respective predetermined relaxation time duration;
and wherein the respective predetermined relaxation time duration of each instance of the second mode of operation is at least five (5) minutes.

7. A method for mitigating or preventing formation of pressure ulcers in a patient, comprising:
transmitting a plurality of intermittent transmissions of an electrical stimulus, each one of the plurality of intermittent transmissions sufficient to effect contraction of a muscle, to a skin portion of a patient, thereby effecting contraction of the muscle;
wherein a respective time interval is provided between each pair of successive intermittent transmissions of the plurality of intermittent transmissions, such that there is a plurality of respective time intervals;
and wherein the muscle is relaxed during the entirety, or the substantial entirety, of each one of the plurality of respective time intervals;
and wherein each one of the plurality of respective time intervals is at least five (5) minutes.

8. Use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein a respective time interval is provided between each pair of successive intermittent transmissions of the plurality of intermittent transmissions, such that there is a plurality of respective time intervals, and wherein the muscle is relaxed during the entirety, or the substantial entirety, of each one of the plurality of respective time intervals, and wherein each one of the plurality of respective time intervals is at least five (5) minutes.

9. A method for mitigating or preventing formation of pressure ulcers in a patient by transmitting an electrical stimulus to a skin portion of a patient sufficient to effect contraction of a muscle, wherein the method includes a first mode of operation and a second mode of operation, comprising:
over a period of at least about one hour, continuously switching between the first mode of operation and the second mode of operation;
wherein, for each instance of the first mode of operation, the first mode of operation lasts a respective predetermined stimulus time duration, and a respective operative electrical stimulus, sufficient to effect contraction of a muscle, is transmitted to a skin portion by an electrode, in contact with the skin portion, thereby effecting contraction of the muscle, during the entire, or substantially the entire, respective predetermined stimulus time duration;
and wherein, for each instance of the second mode of operation, the second mode of operation lasts a respective predetermined relaxation time duration, and no electrical stimulus, or substantially no electrical stimulus, is transmitted to the skin portion by the electrode during the entire, or substantially the entire, respective predetermined relaxation time duration.

10. The method as claimed in claim 9, wherein the respective predetermined relaxation time duration for each instance of the second mode of operation is at least five (5) minutes.

11. A method for mitigating or preventing formation of pressure ulcers in a patient, comprising:
over a period of at least about one hour, and by way of an electrode in contact with a skin portion of the patient, transmitting a plurality of intermittent transmissions of an electrical stimulus, each one of the plurality of intermittent transmissions sufficient to effect contraction of a muscle, to the skin portion of the patient, thereby effecting contraction of the muscle;
wherein a respective time interval is provided between each pair of successive intermittent transmissions of the plurality of intermittent transmissions, such that there is a plurality of respective time intervals;
and wherein no electrical stimulus, or substantially no electrical stimulus, is transmitted to the skin portion by the electrode during the entirety, or the substantial entirety, of each one of the plurality of respective time intervals.

12. The method as claimed in claim 11, wherein each one of the plurality of respective time intervals is at least five (5) minutes.

13. Use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions is transmitted over a period of at least about one hour by an electrode in contact with the skin portion, and wherein a respective time interval is provided between each pair of successive intermittent transmissions of the plurality of intermittent transmissions, such that there is a plurality of respective time intervals, and wherein no electrical stimulus, or substantially no electrical stimulus, is transmitted to the skin portion by the electrode during the entirety, or the substantial entirety, of each one of the plurality of respective time intervals.

14. A method for mitigating or preventing formation of pressure ulcers in a patient by transmitting an electrical stimulus to a skin portion of a patient sufficient to effect contraction of a muscle, wherein the method includes a first mode of operation and a second mode of operation, comprising:
continuously switching between the first mode of operation and the second mode of operation;
wherein, for each instance of the first mode of operation, the first mode of operation lasts a respective predetermined stimulus time duration, and a respective operative electrical stimulus, sufficient to effect contraction of a muscle, is transmitted to the skin portion by an electrode, in contact with the skin portion, thereby effecting contraction of the muscle, during the entire, or substantially the entire, respective predetermined stimulus time duration;
and wherein, for each instance of the second mode of operation, the second mode of operation lasts a respective predetermined relaxation time duration, and the muscle is relaxed during the entire, or substantially the entire, respective predetermined relaxation time duration;
and wherein the respective predetermined relaxation time duration of each instance of the second mode of operation is at least five (5) minutes.

15. A method for mitigating or preventing formation of pressure ulcers in a patient, comprising:
transmitting a plurality of intermittent transmissions of an electrical stimulus with an electrode in contact with a skin portion of the patient, each one of the plurality of intermittent transmissions sufficient to effect contraction of a muscle, to the skin portion, thereby effecting contraction of the muscle;
wherein a respective time interval is provided between each pair of successive intermittent transmissions of the plurality of intermittent transmissions, such that there is a plurality of respective time intervals;
and wherein no electrical stimulus, or substantially no electrical stimulus, is transmitted to the skin portion by the electrode during the entirety, or the substantial entirety, of each one of the plurality of respective time intervals;
and wherein each one of the plurality of respective time intervals is at least five (5) minutes.

16. Use of a plurality of intermittent transmissions of an operative electrical stimulus, sufficient to effect contraction of a muscle, to a skin portion of a patient, thereby effecting contraction of the muscle, for mitigating or preventing formation of pressure ulcers in the patient, wherein the plurality of intermittent transmissions is transmitted by an electrode in contact with the skin portion, and wherein a respective time interval is provided between each pair of successive intermittent transmissions of the plurality of intermittent transmissions, such that there is a plurality of respective time intervals, and wherein no electrical stimulus, or substantially no electrical stimulus, is transmitted to the skin portion by the electrode during the entirety, or the substantial entirety, of each one of the plurality of respective time intervals, and wherein each one of the plurality of respective time intervals is at least five (5) minutes.

17. A method for mitigating or preventing formation of pressure ulcers in a patient comprising effecting a treatment, wherein the treatment is intermittently transmitting an electrical stimulus to a skin portion of a patient sufficient to effect contraction of a muscle, and thereby effecting contraction of the muscle, wherein the muscle being contracted is not pre-conditioned immediately prior to the treatment.

18. The method as claimed in claim 17, wherein during the 60 day period immediately prior to the treatment, the mass of the muscle intended to be contracted during the treatment increases less than about 5%.

19. The method as claimed in claim 17, wherein during the 60 day period immediately prior to the treatment, the endurance of the muscle intended to be contracted during the treatment increases less than about 5%.

20. Use of an intermittent electrical stimulus being transmitted to a skin portion of a patient and sufficient to effect contraction of a muscle, and thereby effecting contraction of the muscle to effect a treatment for mitigating or preventing formation of pressure ulcers in the patient, wherein the muscle being contracted is not pre-conditioned immediately prior to the treatment.

21. A method for mitigating or preventing formation of pressure ulcers in a patient comprising intermittently transmitting an electrical stimulus to a skin portion of a patient sufficient to effect contraction of a muscle, and thereby effecting contraction of the muscle, wherein the electrical stimulus being transmitted is insufficient to effect lifting of the patient or movement of the limbs.

22. The method as claimed in claim 21, wherein the electrical stimulus effects isometric contraction of the muscle.

23. The method as claimed in claim 21, wherein the electrical stimulus effects a change in the angle between two bones defining each and every joint of the patient by less than 10 degrees.

24. Use of an intermittent electrical stimulus being transmitted to a skin portion of a patient and sufficient to effect contraction of a muscle, and thereby effecting contraction of the muscle in order to mitigate or prevent formation of pressure ulcers in the patient, wherein the electrical stimulus being transmitted is insufficient to effect lifting of the patient or movement of the limbs.

25. A method for mitigating or preventing formation of pressure ulcers in a patient comprising intermittently transmitting an electrical stimulus to a skin portion of a patient sufficient to effect contraction of a muscle, and thereby effecting contraction of the muscle, wherein the patient is disposed in a supine position or in a recumbence position when the skin portion is receiving the electrical stimulus.

26. Use of an electrical stimulus being transmitted to a skin portion of a patient sufficient to effect contraction of a muscle, and thereby effecting contraction of the muscle in order to mitigate or prevent formation of pressure ulcers in the patient, wherein the patient is disposed in a supine position or in a recumbence position when the skin portion is receiving the electrical stimulus
